(19) United States
(12) Patent Application Publication
(10) Pub. No.: US 2005/0272755 A1
(43) Pub. Date: Dec. 8, 2005

(54) Method for treating abnormal cell growth

(75) Inventors: Louis J. Denis, Pawcatuck, CT (US); Linda D. Compton, Richland, MI (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/145,097

(22) Filed: Jun. 3, 2005

Related U.S. Application Data
(60) Provisional application No. 60/577,268, filed on Jun. 4, 2004.

(51) Int. Cl. A61K 31/4745

(52) U.S. Cl. 514/283

(57) ABSTRACT

The present Invention relates to a method of treating abnormal cell growth in a subject, comprising administering to said subject having abnormal cell growth: (a) a compound selected from the group consisting of a camptothecin, a camptothecin derivative, or a pharmaceutically acceptable salt, solvate or prodrug of said compounds; (b) a pyrimidine derivative or a pharmaceutically acceptable salt, solvate or prodrug of said pyrimidine derivative; and (c) an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, anti-androgens and combinations thereof.

### FIELD OF THE INVENTION

The present Invention relates to a method of treating abnormal cell growth in a subject, comprising administering to said subject having abnormal cell growth: (a) a compound selected from the group consisting of a camptothecin, a camptothecin derivative, an indolopyrrocarbazole derivative, or a pharmaceutically acceptable salt, solvate or prodrug of said compounds; (b) a pyrimidine derivative or a pharmaceutically acceptable salt, solvate or prodrug of said pyrimidine derivative; and (c) an anti-tumor agent.

### BACKGROUND OF THE INVENTION

The invention relates to the treatment of abnormal cell growth, e.g., cancer, especially solid tumors, with combinations of (i) a camptothecin, camptothecin derivatives or indolopyrrocarbazole derivatives, (ii) pyrimidine derivatives and (iii) other anticancer drugs.

Colorectal cancer is a leading cause of morbidity and mortality with about 300,000 new cases and 200,000 deaths in Europe and the USA each year (See P. Boyle, Some Recent Developments in the Epidemiology of Colorectal Cancer, pages 19-34 in Management of Colorectal Cancer, Bleiberg H., Rougier P., Wilke H. J., eds, (Martin Dunitz, London 1998); and—Midgley R. S., Kerr D. J., Systemic Adjuvant Chemotherapy for Colorectal Cancer, pages 126-27 in Management of Colorectal Cancer, Bleiberg H., Rougier P., Wilke H. J., eds, (Martin Dunitz, London 1998).) Although about fifty percent of patients are cured by surgery alone, the other half will eventually die due to metastatic disease, which includes approximately twenty-five percent of patients who have evidence of metastases at time of diagnosis.

5-FU is an intravenously (IV) administered fluorinated pyrimidine cytotoxic agent that inhibits the function of thymidylate synthase, an enzyme necessary for the production of the thymidine nucleotides required for DNA synthesis. 5-FU has activity in the therapy of a number of tumor types but is most commonly given in the treatment of colorectal cancer, upper gastrointestinal malignancies, and breast cancer. In the therapy of colorectal cancer, 5-FU is customarily administered with the biomodulating agent, leucovorin (LV), which acts to facilitate affinity with thymidylate synthase, thereby improving 5-FU efficacy (Grem J L. 5-Fluoropyrimidines. In: Cancer Chemotherapy and Biotherapy: Principles and Practice, 2^{nd} ed, Chabner B A and Longo D L, eds, Lippincott-Raven Publishers, Philadelphia; pp. 149-211, 1996). Erratic oral bioavailability has historically mandated IV administration of 5-FU (Hahn R G, Moertel C G, Schutt A J, et al. A double-blind comparison of intensive course 5-fluorouracil by oral vs IV route in the treatment of colorectal carcinoma. Cancer 35:1031-1035, 1975).

Like 5-FU, CPT-11 (Irinotecan Hydrochloride, Irinotecan Hydrochloride Hydrate, Camptosar®) is a semi-synthetic derivative of camptothecin and had broad-spectrum cytotoxic activity. CPT-11 has been primarily been developed for use in the therapy of colorectal cancer. CPT-11 is a prodrug that is administered IV and is metabolized by carboxylesterases in human liver, tumors, and other tissues to the more active lipophilic metabolite, SN-38 (Tsuji T, Kaneda N, Kado K, et al. CPT-11 converting enzyme from rat serum: purification and some properties. J Pharmacobiodyn 1992; 14: 341-349). SN-38 functions as an inhibitor of topoisomerase 1, a nuclear enzyme that plays a critical role in DNA replication and transcription (Pommier Y, Tanizawa A, Kohn K W. Mechanisms of topoisomerase I inhibition by anticancer drugs. In: Liu L F, ed. Advances in Pharmacology. New York: Academic Press; 29B:73-92, 1994). The enzyme functions normally to cause transient breaks in a single strand of DNA that release the torsional strain caused by synthesis of a new strand of DNA or RNA around the double helix. SN-38 targets this topoisomerase I-DNA complex, stabilizing it and inhibiting reannealing of the parent DNA. Collision of replication forks with the stabilized complex during cell division leads to double-stranded DNA breaks and tumor cell death.

In colorectal cancer patients resistant to 5-FU, single agent CPT-11 tested in two large phase III randomized trials resulted in a longer survival and a better quality of life compared with supportive care only (D. Cunningham, S. Pyrhonen, R D. James et al, The Lancet, 352 (9138):1413-1418 (1998)) and also in a longer survival without deterioration in quality of life compared with 5-FU/FA best infusional regimens (P. Rougier, E. van Cutsem et al; The Lancet, 352 (9138):1407-1418 (1998)). CPT-11 is therefore the reference treatment in metastatic colorectal cancer (MCRC) after failure on prior 5-FU treatment.

The combination of 5-FU/LV with CPT-11 has been registered as therapy of colorectal cancer based on randomized clinical trial data documenting that this combination can significantly improve tumor response rates, lengthen time to tumor progression, and prolong survival.

The oral administration of cell-cycle-specific agents such as the fluoropyrimidines or irinotecan is an attractive alternative to IV administration of these types of agents. Oral formulations can achieve protracted drug exposure to actively cycling malignant cells at a time of greatest vulnerability without the need for continuous IV infusion. An oral formulation may offer the advantages of patient convenience and a less expensive means of prolonged drug administration.

A method that has been used to overcome the poor oral bioavailability of 5-FU involves the administration of a prodrug that has good bioavailability and is ultimately converted to 5-FU. Capecitabine (N⁴-pentyloxycarbonyl-5′-deoxy-5-fluorocytidine, Xeloda®) is such a novel oral fluoropyrimidine carbamate. It is readily absorbed from the gastrointestinal tract and is preferentially converted to 5-FU in tumor Ussue. After oral administration, capecitabine passes intact from the gastrointestinal tract to the liver, where it is converted by carboxylesterases to 5′-deoxy-5-flourocytidine (5′-DFCR), then by cytidine deaminase in liver and tumor tissue to 5′-deoxy-5-flourouridine (5′-DFUR), and finally by thymidine phosphorylase (dThdPase) in tumor tissue to 5-FU.

The recommended phase II, single-agent dose of capecitabine is 2500 mg/m²/day for 14 days every 3 weeks [Mackean M. Planting A, Twelves, J, et al. Phase I and pharmacologic study of intermittent twice-daily oral therapy with capecitabine in patients with advanced and/or metastatic cancer. J Clin Oncol 16(9):2977-2985, 1998; Van Cutsem E, Findlay M, Osterwalder B, et al. Capecitabine, an oral fluoropyrimidine carbamate with substantial activity in advanced colorectal cancer: Results of a randomized phase 11 study. J Clin Oncol 18(6):1337-1345, 2000]. Two phase III trials have shown capecitabine to have activity in advanced colon cancer that is comparable to 5-FU. The 2 trials, in previously untreated patients with metastatic colorectal cancer, were conducted in Europe (N=602) and in the US (N=605) comparing single-agent capecitabine (2500 mg/m² day for 14 days every 3 weeks) to IV 5-FU/LV (Mayo Clinic regimen) (Twelves C, Harper P, Van Cutsem E, et al. A phase III trial (S014796) of Xeloda (capecitabine) in previously untreated advanced/metastatic colorectal cancer. Proc Am Soc Clin Oncol 1999; 18:263a (abstract 1010); Cox J, Pazdur R, Thibault A, et al. A phase III trial of Xeloda (capecitabine) in previously untreated advanced/metastatic colorectal cancer. Proc Am Soc Clin Oncol 1999; 18:265a (abstract 1016).). Overall response rates were significantly greater in the capecitabine treatment arms (21% for both studies) compared to the IV 5-FU/LV treatment arms (11% and 14%, respectively) (p=0.014 and 0.03, respectively). Duration of response and time to tumor progression were similar between treatment groups in both trials. Furthermore, overall survival was similar in the capecitabine and IV 5-FU/LV treatment groups in both trials (Xeloda. Hoffman-LaRoche Limited, Mississauge, Ontario. Product Monograph: Jul. 5, 2000). In the US trial, the median survival was 12.5 months for patients treated with capecitabine compared to 13.4 months for patients treated with IV 5-FU/LV (p=0.24). In the European trial, the median survival was 13.3 months versus 12.5 months for the capecitabine and IV 5-FU/LV patients, respectively (p=0.30). The most common grade 3-4 toxicities reported in the capecitabine treatment arms were hand-foot syndrome (17%) and diarrhea (14%).

Other toxicities associated with the use of capecitabine include myelosuppression, transient hyperbilirubinemia, fatigue, dehydration, nausea, vomiting, stomatitis, abdominal pain, constipation, nosebleed, dermatitis, anorexia, pyrexia, paraesthesia, headache, dizziness, insomnia, eye irritation, myalgia, and edema.

Initial studies to develop an oral irinotecan commenced with a phase I study of the IV irinotecan formulation mixed with 50 mL of CranGrape® juice. Study treatment was administered orally once per day for 5 days every 3 weeks to 28 patients (Drengler R L, Kuhn J G, Schaaf L J, et al. Phase I and pharmacokinetic trial of oral irinotecan administered daily for 5 days every 3 weeks in patients with solid tumors. J Clin Oncol (17):685-696, 1999). As with IV irinotecan, grade 4 delayed diarrhea proved to be dose limiting. Although patient cohorts were small, there appeared to be the possibility of an age-related variation in the extent of dose-limiting diarrhea; excessive proportions of patients experiencing this dose-limiting toxicity (DLT) at the 80-mg/m²/day dosage in patients <65 years of age and at the 66-mg/m²/day dosage in patients ≧65 years of age. The maximum tolerated doses (MTDs) and recommended phase II starting doses for oral irinotecan was therefore considered to be 66 mg/m²/day in patients <65 years and 50 mg/m²/day in patients ≧65 years. Several objective tumor responses were observed in patients with colorectal cancer, documenting that oral administration of irinotecan could provide antineoplastic activity.

Following the preliminary study of IV irinotecan given orally (Protocol M/6475/0032), 4 phase 1, single-agent, dose-finding trials of a finished Powder Filled Capsules (PFC) formulation of irinotecan were initiated; 2 studies have been conducted in Europe (Protocols CPT X 117 and CPT X 118) by Aventis and 2 studies have been performed in the US (Protocols 139 and 155) by Pharmacia (now part of Pfizer Inc.). Protocols 117 and 139 are studying a 5-day every 3-week schedule and Protocols 118 and 155 are studying a 14-day every 3-week schedule. These studies have found that irinotecan can be given orally, and when administered either as an IV solution given orally or as a PFC formulation, has shown a tolerable safety profile and antitumor activity in phase I studies. However, the PFC formulations are not very desirable due to the handling concerns especially during the manufacturing process which can expose manufacturing workers to undesirable toxic exposure to the drug. Furthermore, there are concerns that PFC give rise to a higher risk of harm to patients on drug due patient mishandling of the drug (e.g., breakage of PFC capsule), as well as other non-treated individuals who come into contact with the PFC capsules (or broken capsules or spilled drug), such as other family members, doctors and pharmacists. Applicants have developed a new formulation for oral irinotecan which solves these problems. The new formulation is a semi-solid matrix (SSM) formulation of oral irinotecan provides similar preclinical bioavailability as the PFS formulation and offers improved handling characteristics. Additionally, applicants have found that combination of capecitabine and oral irinotecan (SSM) is an effective treatment in patients with advanced solid tumors.

The oral formulation of irinotecan has particularly utility in developing of all oral cancer treatment regiments for combination therapy with other agents such as pyrimidine derivatives such as capecitabine and other anti-tumor agents.

### SUMMARY OF THE INVENTION

The present invention relates to a method of treating abnormal cell growth in a subject, comprising administering to said subject having abnormal cell growth: (a) a compound selected from the group consisting of a camptothecin, a camptothecin derivative, the indolopyrrocarbazole derivative, or a pharmaceutically acceptable salt, solvate or prodrug of said compounds; (b) a pyrimidine derivative or a pharmaceutically acceptable salt, solvate or prodrug of said pyrimidine derivative; and (c) an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In one embodiment of the present invention the camptothecin or camptothecin derivative is selected from the group consisting of camptothecin, 10-hydroxycamptothecin, 9-aminocamptothecin, 9-nitrocamptothecin, irinotecan, irinotecan salt, SN-38, CPT-11, topotecan or a pharmaceutically acceptable salt, solvate or prodrug thereof and the indolopyrrocarbazole derivative is edotercarin.

In a preferred embodiment the camptothecin derivative is selected from the group consisting of irinotecan, SN-38, topotecan or a pharmaceutically acceptable salt, solvate or prodrug thereof.

In one more preferred embodiment of the present invention the camptothecin derivative is irinotecan.

In another more preferred embodiment of the present invention the camptothecin derivative is a pharmaceutically acceptable salt of irinotecan.

In an even more preferred embodiment of the present invention the camptothecin derivative is a hydrochloride salt of irinotecan.

In another even more preferred embodiment of the present invention the camptothecin derivative is irinotecan hydrochloride trihydrate.

In a most preferred embodiment the camptothecin derivative is CPT-11.

In another embodiment of the present invention the camptothecin derivative is SN-38 and prodrugs thereof.

In one embodiment of the present invention the camptothecin derivative is administered orally.

In another embodiment of the present invention the camptothecin derivative is topotecan.

In one preferred embodiment of the present invention the pyrimidine derivative is selected from the group consisting gemcitabine, multitargeted antifolate (Alimta, MTA) and capecitabine.

In one preferred embodiment of the present invention the pyrimidine derivative is selected from the group consisting gemcitabine and capecitabine.

In a more preferred embodiment of the present invention the pyrimidine derivative is gemcitabine.

In a most preferred embodiment of the present invention the pyrimidine derivative is capecitabine.

In one embodiment of the present invention the pyrimidine derivative is administered orally.

In one embodiment of the present invention the camptothecin, the camptothecin derivative, the indolopyrrocarbazole derivative, the pharmaceutically acceptable salt, solvate or prodrug of said compounds is administered orally.

In one embodiment of the present invention the anti-tumor agent is selected from the group consisting of SU-11248, CP-547,632, CP-868,596, CP-724,714, CI-1033, GW-572016, pan erbB2 inhibitor, CTLA4 monoclonal antibody, IGF1R monoclonal antibody, CD40 monoclonal antibody, AG-013736, AG-002037, PD-0332991, PD-0325901, Aromasin® (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Omnitarg, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125, Arcoxia (etoricoxib) and radiation.

In a preferred embodiment of the present invention the anti-tumor agent is selected from the group consisting of SU-11248, CP-547,632, CP-868,596, CP-724,714, C₁₋₁₀₃₃, GW-572016, AG-013736, AG-002037, PD-0332991, and PD-0325901.

In a preferred embodiment of the present invention the anti-tumor agent is selected from the group consisting of SU-11248, CP-547,632, CP-868,596, GW-572016, and CP-724,714.

In a preferred embodiment of the present invention the anti-tumor agent is selected from the group consisting of AG-013736, AG-002037, PD-0332991, and PD-0325901.

In a preferred embodiment of the present invention the the anti-tumor agent is Aromasin® (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125, Arcoxia (etoricoxib) and radiation.

In a more preferred embodiment of the present invention the anti-tumor agent is Tarceva™ (erlotinib HCl), Avastin™ (bevacizumab), Erbitux™ (Cetuximab or C225), Herceptin®, Omnitarg, Gleevec® (imatinib mesylate) and Iressa™ (genfitinib).

In a more preferred embodiment of the present invention the anti-tumor agent is Aromasin® (exemstane), Ellence® (epirubicin), Zinecard® (dexrazoxane), Taxol® (paclitaxel), and Adriamycin® (doxorubicin).

In a preferred embodiment of the present invention the anti-tumor agent is Celebrex® (celecoxib), paracoxib, paracoxib, Vioxx® (rofecoxib), Bextra® (valdecoxib), and Arcoxia™ (etoricoxib).

In a more preferred embodiment of the present invention the anti-tumor agent is selected from the group consisting of Tarceva™ (erlotinib HCl), Avastin™ (bevacizumab), Erbitux™ (Cetuximab or C225), Omnitarg, and Herceptin®.

In a more preferred embodiment of the present invention the anti-tumor agent is selected from the group consisting of Tarceva™ (erlotinib HCl) and Avastin™ (bevacizumab).

In a most preferred embodiment of the present invention the anti-tumor agent is Tarceva™ (erlotinib HCl).

In a most preferred embodiment of the present invention the anti-tumor agent is SU-11248.

In a most preferred embodiment of the present invention the anti-tumor agent is Avastin™ (bevacizumab).

In a most preferred embodiment of the present invention the anti-tumor agent is Erbitux™ (Cetuximab or C225).

In a most preferred embodiment of the present invention the antitumor agent is radiation.

In one preferred embodiment the antitumor agent is gamma radiation.

In one more preferred embodiment of the present invention 14 GY radiation is administered.

In another more preferred embodiment of the present invention 10 GY radiation is administered.

In another more preferred embodiment of the present invention 7 GY radiation is administered.

In one preferred embodiment of the present invention the compounds (a), (b) and (c) are administered simultaneously, semi-simultaneously, separately, or sequentially during a treatment cycle.

In one more preferred embodiment of the present invention compounds (a), (b) and (c) are administered simultaneously or semi-simultaneously during a treatment cycle.

In one more preferred embodiment of the present invention compounds (a), (b) and (c) are administered separately or sequentially during a treatment cycle.

In one embodiment of the present invention abnormal cell growth is cancer is selected from the group consisting of mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In one more preferred embodiment of the present invention abnormal cell growth is cancer selected from the group consisting of breast, lung (NSCLC and SCLC), gastrointestinal (gastric, colorectal, and duodenal), pancreatic, hepatobilliary (hepatic, and billiary duct), a primary or secondary CNS tumor, and malignant melanoma.

In one even more preferred embodiment of the present invention abnormal cell growth is cancer is selected from the group consisting of breast, lung (NSCLC and SCLC), a primary or secondary CNS tumor, and malignant melanoma.

In one most preferred embodiment of the present invention abnormal cell growth is a cancer selected from the group consisting of breast, and non-small cell lung and small cell lung.

In one embodiment of the present invention abnormal cell growth is a cancer which is metastatic or early cancer.

In another embodiment of the present invention treatment is administered in the neoadjuvant setting, adjuvant setting, or in the metastatic disease setting.

In one embodiment of the present invention is directed to a method of treating cancer in a subject, comprising administering to said subject having cancer oral CPT-11, capecitabine, and an anti-tumor agent selected from the group consisting of SU-11248, CP-547,632, CP-868,596, CP-724,714, CI-1033, GW-572016, pan erbB2 inhibitor, CTLA4 monoclonal antibody, IGF1R monoclonal antibody, CD40 monoclonal antibody, AG-013736, AG-002037, PD-0332991, PD-0325901, Aromasin® (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Omnitarg, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125, Arcoxia (etoricoxib) and radiation.

In preferred embodiment of the present invention the anti-tumor agent is selected from the group consisting of SU-11248, CP-547,632, CP-868,596, GW572016, Tarceva™ (erlotinib HCl), Avastin™ (bevacizumab), Erbitux™ (Cetuximab or C225), Celebrex® (celecoxib), paracoxib, paracoxib, Herceptin®, Omnitarg, Vioxx®, (rofecoxib), Bextra® (valdecoxib), Arcoxia™ (etoricoxib) and radiation.

In one more preferred embodiment of the present invention the anti-tumor agent is selected from the group consisting of SU-11248, GW572016, Tarceva™ (erlotinib HCl), Avastin™ (bevacizumab), Erbitux™ (Cetuximab or C225), Herceptin®, and radiation.

In an even more preferred embodiment of the present invention the anti-tumor agent is selected from the group consisting of SU-11248, Tarceva™ (erlotinib HCl), Herceptin®, Avastin™ (bevacizumab) and radiation.

In one even more preferred embodiment of the present invention the anti-tumor agent is selected from the group consisting of SU-11248, Tarceva™ (erlotinib HCl) and radiation.

In one more preferred embodiment of the present invention 40 to 50 mg/m² of oral CPT-11 is administered on days 1 to 5 of a three week cycle and 800 to 1250 mg/m² of the capecitabine is administered on days 6 to 14 of the three week cycle.

In one more preferred embodiment of the present invention the third week of the cycle is drug free.

In one more preferred embodiment of the present invention the oral CPT-11 is administered once a day.

In one more preferred embodiment of the present invention the capecitabine is administered twice a day.

In one more preferred embodiment of the present invention capecitabine is orally administered twice a day.

In one more preferred embodiment of the present invention relates to a method of treating cancer in a subject, comprising administering to said subject having cancer CPT-11, capecitabine, and radiation.

In one more preferred embodiment of the present invention CPT-11 is administered orally.

In one more preferred embodiment of the present invention capecitabine is administered orally.

In one more preferred embodiment of the present invention the CPT-11, capecitabine and radiation are administered sequentially or separately in any order.

In another more preferred embodiment of the present invention 40 to 50 mg/m² of the oral CPT-11 is administered on days 1 to 5 of a three week cycle and 800 to 1000 mg/m² of the capecitabine is administered on days 6 to 14 of the three week cycle. In one embodiment of the present invention relates to a method of treating a mammal having a cancer, comprising: administering to said mammal in need of such treatment, sequentially or separately in either order, (i) a therapeutically effective amount of an oral camptothecin, an oral camptothecin derivative, an indolopyrrocarbazole derivative or a pharmaceutically acceptable salt, solvate or prodrug thereof, (ii) a therapeutically effective amount of a pyrimidine derivative or a pharmaceutically acceptable salt, solvate or prodrug thereof, and (iii) a therapeutically effective amount of an anti-tumor agent.

In one preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered separately or sequentially during a regimen, a cycle, a schedule or a course.

In one more preferred embodiment of the invention the oral camptothecin, the oral camptothecin derivative, the indolopyrrocarbazole derivative or the pharmaceutically acceptable salt, solvate or prodrug thereof the pyrimidine derivative or pharmaceutically acceptable salt, solvate or prodrug thereof, and the anti-tumor agent are administered separately or sequentially in any order during a regimen.

In one more preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered separately or sequentially in any order during a cycle.

In one more preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered separately or sequentially in any order during a schedule

In one more preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered separately or sequentially in any order during a course

In one more preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered separately in any order.

In one more preferred embodiment of the invention the oral camptothecin, the oral camptothecin derivative, the indolopyrrocarbazole derivative or the pharmaceutically acceptable salt, solvate or prodrug thereof, the pyrimidine derivative or pharmaceutically acceptable salt, solvate or prodrug thereof, and the anti-tumor agent are administered sequentially in any order.

In one preferred embodiment of the invention the combination of oral camptothecin, and oral camptothecin derivative, pyrimidine derivative and anti-tumor agent are therapeutically effective for treating said cancer.

In one preferred embodiment of the invention an amount of 40 to 50 mg/m² of oral CPT-11 is administered once a day.

In one more preferred embodiment of the invention an amount of 40 to 45 mg/m² of oral CPT-11 is administered once a day.

In a most preferred embodiment of the invention an amount of 40 mg/m² of oral CPT-11 is administered once a day.

In another most preferred embodiment of the invention an amount of 50 mg/m² of oral CPT-11 is administered once a day.

In one preferred embodiment of the invention an amount of 800 to 1250 mg/m² of capecitabine is administered twice a day.

In a more preferred embodiment of the invention an amount of 800 to 1000 mg/m² of capecitabine is administered twice a day.

In a most preferred embodiment of the invention an amount of 800 mg/m² of capecitabine is administered twice a day.

In another most preferred embodiment of the invention an amount of 1000 mg/m² of capecitabine is administered twice a day.

In one preferred embodiment of the invention the oral camptothecin, or oral camptothecin derivative, pyrimidine derivative, and anti-tumor agent are administered separately or sequentially during a regimen, a cycle, a schedule or a course.

In one more preferred embodiment of the invention the oral camptothecin, or oral camptothecin derivative, pyrimidine derivative and anti-tumor agent are administered separately or sequentially during a regimen.

In one more preferred embodiment of the invention the oral camptothecin, or oral camptothecin derivative, pyrimidine derivative and anti-tumor agent are administered separately or sequentially during a cycle.

In one more preferred embodiment of the invention the oral camptothecin, or oral camptothecin derivative, pyrimidine derivative and anti-tumor agent are administered separately or sequentially during a schedule.

In one more preferred embodiment of the invention the oral camptothecin, or oral camptothecin derivative, and pyrimidine derivative and anti-tumor agent are administered separately or sequentially during a course.

In one preferred embodiment of the invention the oral camptothecin, or oral camptothecin derivative, pyrimidine derivative and anti-tumor agent are administered separately.

In one preferred embodiment of the invention the oral camptothecin, or oral camptothecin derivative, pyrimidine derivative and anti-tumor agent are administered sequentially.

In one preferred embodiment of the invention the oral camptothecin, or oral camptothecin derivative, pyrimidine derivative, and anti-tumor agent are administered semi-simultaneously or simultaneously during a regimen, a cycle, a schedule or a course.

In one more preferred embodiment of the invention the oral camptothecin, or oral camptothecin derivative, pyrimidine derivative and anti-tumor agent are administered semi-simultaneously or simultaneously during a regimen.

In one more preferred embodiment of the invention the oral camptothecin, or oral camptothecin derivative, pyrimidine derivative and anti-tumor agent are administered semi-simultaneously or simultaneously during a cycle.

In one more preferred embodiment of the invention the oral camptothecin, or oral camptothecin derivative, pyrimidine derivative and anti-tumor agent are administered semi-simultaneously or simultaneously during a schedule.

In one more preferred embodiment of the invention the oral camptothecin, or oral camptothecin derivative, and pyrimidine derivative and anti-tumor agent are administered semi-simultaneously or simultaneously during a course.

In one preferred embodiment of the invention the oral camptothecin, or oral camptothecin derivative, pyrimidine derivative and anti-tumor agent are administered semi-simultaneously.

In one preferred embodiment of the invention the oral camptothecin, or oral camptothecin derivative, pyrimidine derivative and anti-tumor agent are administered simultaneously.

In one preferred embodiment of the invention the cycle is one or more treatment cycles.

In one preferred embodiment of the invention each of the one or more treatment cycles is at least three weeks in duration.

In one preferred embodiment of the invention the oral CPT-11 is administered for five days during the cycle.

In one preferred embodiment of the invention the five days of oral CPT-11 administration during the cycle are consecutive.

In one preferred embodiment of the invention the oral CPT-11 is administered during day 1 through day 5 of the treatment cycle.

In one preferred embodiment of the invention the capecitabine is administered during nine days of the treatment cycle.

In one preferred embodiment of the invention the nine days of Capecitabine administration during the treatment cycle are consecutive.

In one preferred embodiment of the invention the capecitabine is administered during day 6 through day 14 of the treatment cycle.

In one preferred embodiment of the invention the oral camptothecin derivative is administered as an encapsulated semi-solid matrix formulation.

In one preferred embodiment of the invention the encapsulated semi-solid matrix formulation is in a capsule.

In one preferred embodiment of the invention the oral camptothecin derivative administered in an encapsulated semi-solid matrix formulation is CPT-11.

In one more preferred embodiment of the invention the semi-solid matrix formulation comprises Geluire and Lecithin.

In one preferred embodiment of the invention the pyrimidine derivative is administered as an oral dosage form.

In one preferred embodiment of the invention the pyrimidine derivative is capecitabine.

In one preferred embodiment of the invention the at least three week treatment cycle is a drug free of oral camptothecin and an oral camptothecin derivative selected from the group consisting of 10-hydroxycamptothecin, 9-aminocamptothecin, 9-nitrocamptothecin, irinotecan, irinotecan salt, SN-38, CPT-11, and topotecan and pyrimidine derivative and anti-tumor agent.

In one preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capecitabine, and (iii) a therapeutically effective amount of an anti-tumor agent.

In one preferred embodiment the method of the present invention is sequentially administered in any order.

In one preferred embodiment the method of the present invention is separately administered in any order.

In one preferred embodiment the method of the present invention is semi-simultaneously administered.

In one preferred embodiment the method of the present invention is simultaneously administered.

In a more preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capecitabine, and (iii) a therapeutically effective amount of an anti-tumor agent selected from the group consisting of SU-11248, CP-547,632, CP-868,596, CP-724,714, CI-1033, GW-572016, pan erbB2 inhibitor, CTLA4 monoclonal antibody, IGF1R monoclonal antibody, CD40 monoclonal antibody, AG-013736, AG-002037, PD-0332991, PD-0325901, Aromasin®) (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Omnitarg, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125, Arcoxia (etoricoxib) and radiation.

In a more preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capedtabine, and (iii) a therapeutically effective amount of an anti-tumor agent selected from the group consisting of SU-11248, CP-547,632, CP-868,596, GW572016, Tarceva™ (erlotinib HCl), Avastin™ (bevacizumab), Erbitux™ (Cetuximab or C225), Celebrex® (celecoxib), paracoxib, Herceptin®, Omnitarg, Vioxx®, (rofecoxib), Bextra® (valdecoxib), Arcoxia™ (etoricoxib) and radiation.

In a more preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capecitabine, and (iii) a therapeutically effective amount of an anti-tumor agent is selected from the group consisting of SU-11248, GW572016, Tarceva™ (erlotinib HCl), Avastin™ (bevacizumab), Erbitux™ (Cetuximab or C225), Herceptin®, and radiation.

In a more preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capecitabine, and (iii) a therapeutically effective amount of an anti-tumor agent is selected from the group consisting of SU-11248, Tarceva™ (erlotinib HCl), Herceptin®, Avastin™ (bevacizumab) and radiation.

In an even more preferred embodiment the method of the invention comprises treating a mammal having a cancer, comprising: administering to said mammal in need of such treatment, sequentially, separately, semi-simultaneously, or simultaneously, (i) a therapeutically effective amount of oral CPT-11, (ii) a therapeutically effective amount of Capecitabine, and (iii) a therapeutically effective amount of an anti-tumor agent selected from the group consisting of SU-11248, Tarceva™ (erlotinib HCl) and radiation.

In a most preferred embodiment the method of the invention comprises treating a mammal having a cancer, comprising: administering to said mammal in need of such treatment, sequentially or separately in either order, (i) a therapeutically effective amount of oral CPT-11, (ii) a therapeutically effective amount of Capedtabine, and (iii) a therapeutically effective amount of an anti-tumor agent SU-11248.

In a most preferred embodiment the method of the invention comprises treating a mammal having a cancer, comprising: administering to said mammal in need of such treatment, sequentially or separately in either order, (i) a therapeutically effective amount of oral CPT-11, (ii) a therapeutically effective amount of Capecitabine, and (iii) a therapeutically effective amount of an anti-tumor agent Tarceva™ (erlotinib HCl).

In a most preferred embodiment the method of the invention comprises treating a mammal having a cancer, comprising: administering to said mammal in need of such treatment, sequentially or separately in either order, (i) a therapeutically effective amount of oral CPT-11, (ii) a therapeutically effective amount of Capecitabine, and (iii) a therapeutically effective amount of an anti-tumor agent Avastin™ (bevacizumab).

In a most preferred embodiment the method of the invention comprises treating a mammal having a cancer, comprising: administering to said mammal in need of such treatment, sequentially or separately in either order, (i) a therapeutically effective amount of oral CPT-11, (ii) a therapeutically effective amount of Capecitabine, and (iii) a therapeutically effective amount of an anti-tumor agent Erbitux™ (Cetuximab or C225).

In a preferred embodiment the method of the invention comprises treating a mammal having a cancer, comprising: administering to said mammal in need of such treatment, (i) CPT-11, (ii) Capecitabine, and (iii) an anti-tumor agent selected from the group consisting of CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole and Arcoxia (etoricoxib).

In a more preferred embodiment the method of the invention comprises treating a mammal having a cancer, comprising: administering to said mammal in need of such treatment, (i) CPT-11, (ii) Capecitabine, and (iii) an anti-tumor agent selected from the group consisting of CELEBREX™ (celecoxib), parecoxib, deracoxib, MK-663 (etoricoxib), Bextra (valdecoxib), paracoxib, and Vioxx (rofecoxib).

In an even more preferred embodiment the method of the invention comprises treating a mammal having a cancer, comprising: administering to said mammal in need of such treatment, (i) CPT-11, (ii) Capecitabine, and (iii) an anti-tumor agent selected from the group consisting of CELEBREX™ (celecoxib), parecoxib, Bextra (valdecoxib), paracoxib, and Vioxx (rofecoxib).

In an most preferred embodiment the method of the invention comprises treating a mammal having a cancer, comprising: administering to said mammal in need of such treatment, (i) CPT-11, (ii) Capecitabine, and (iii) an anti-tumor agent selected from the group consisting of parecoxib, Bextra (valdecoxib), and paracoxib.

In an most preferred embodiment the method of the invention comprises treating a mammal having a cancer, comprising: administering to said mammal in need of such treatment, (i) CPT-11, (ii) Capecitabine, and (iii) an anti-tumor agent selected from the group consisting of CELEBREX™ (celecoxib), and Vioxx (rofecoxib).

In a more preferred embodiment the invention relates to a method of treating cancer in a subject, comprising administering sequentially to said subject having cancer oral CPT-11 and capecitabine, wherein 40 to 50 mg/m² of the oral CPT-11 is administered on days 1 to 5 of a three week cycle and 800 to 1250 mg/m² of the capecitabine is administered on days 6 to 14 of the three week cycle and an anti-tumor agent is administered during, before or after the three week cycle.

In one preferred embodiment of the invention the oral CPT-11 is administered once a day.

In one preferred embodiment of the invention the oral CPT-11 is administered twice a day.

In another preferred embodiment of the invention the oral CPT-11 is administered three times a day.

In one preferred embodiment of the invention the capecitabine is administered twice a day.

In another preferred embodiment of the invention the capecitabine is administered three times a day.

In one embodiment of the invention the anti-tumor agent is administered once a day.

In one preferred embodiment of the invention the anti-tumor agent is administered twice a day.

In another preferred embodiment of the invention the anti-tumor agent is administered three times a day.

In one preferred embodiment of the invention the oral CPT-11, capecitabine and anti-tumor agent are administered during a regimen, a cycle; a schedule or a course.

In one more preferred embodiment of the invention the oral CPT-11, capecitabine and anti-tumor agent are administered during a regimen.

In one more preferred embodiment of the invention the oral CPT-11, capecitabine and anti-tumor agent are administered during a cycle.

In one more preferred embodiment of the invention the oral CPT-11, capecitabine and anti-tumor agent are administered during a schedule.

In one more preferred embodiment of the invention the oral CPT-11, capecitabine and anti-tumor agent are administered during a course.

In one preferred embodiment of the invention when the drug cycle is three weeks in duration the third week of the cycle is drug free.

In another embodiment of the present invention relates to therapeutic pharmaceutical compositions comprising an effective amount of a pyrimidine derivative in combination with an effective amount of an oral camptothecin, an oral camptothecin derivative or an indolopyrrocarbazole derivative, and an anti-tumor agent which are useful for the treatment of cancer.

In another aspect the method of the invention is directed to the method of administration of the combination. More particularly the active agents of the combination therapy are administered sequentially in either order. When the active agents are administered sequentially, one skilled in the art will understand that the second agent can be administered some time after the first agent and the third agent can be administered some time after the second agent. The particular period of delay is dependent on the particular pharmacokinetic and formulation parameters of the active agent.

In another aspect of the invention is the minimization of the combination dose. It is frequently the case that the individual dosage regimes for the active agents can lead to undesirable side effects that can potentially lead to a discontinuation of the medication. One particular preferred embodiment of the invention is to reduce the dosage to the minimum dose necessary to treat the cancer. Thus one preferred embodiment is the administration of a combination wherein the amounts of the active agents is less than the efficacious dose than agents alone. Another embodiment of the invention is the administration of a combination that has activity above the activity of each agent alone. Preferred combinations are those in which the combination is synergistic compared to each agent alone. Preferably, the combination is superadditive.

This invention also relates to a kit for treatment of abnormal cell growth, comprising a combination as defined above, and written instructions for administration of all components. In a particular aspect the specific oral camptothecin and camptothecin derivative and its method of administration is described in the written instructions. In another particular aspect of the kit of the invention, the written instructions specify the pyrimidine derivative and describe its method of administration. In another particular aspect of the kit of the invention, the written instructions specify the anti-tumor agent and describe its method of administration. In one embodiment of said kit, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In another embodiment of said kit, said abnormal cell growth is a benign proliferative disease, including, but not limited to, psoriasis, benign prostatic hypertrophy or restinosis.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

Those active compounds of the present combination invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the active compounds of the present combination invention can be substituted for bioisosteric groups, that is, groups that have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of the combinations of the present invention may also exist as tautomers. This invention relates to the use of all such tautomers and mixtures thereof.

The subject matter of the invention also includes isotopically-labelled compounds, and the pharmaceutically acceptable salts, solvates and prodrugs thereof, which are identical to those recited for the active compounds described herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled active compounds of the combinations of this invention and prodrugs thereof can generally be prepared by procedures well known to those skilled in the art.

This invention also encompasses pharmaceutical compositions containing and methods of treating cancer through administering prodrugs of the active compounds of the present combination invention. Active compounds having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of the active compounds. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The terms synergy and synergistic mean that the combination of two or more effectors or active agents is at least greater than the activity of either agent alone and is preferably at least additive in their effect. More preferably, the synergy is greater than additive. Most preferably, the synergy is superadditive. The term “additive” is use to mean that the result of the combination of the two or more effectors or agents is more than the sum of each effector or agent together and preferably at least 10 percent greater than the combination's additive effect. The term “superadditive” is used to mean that the result of combination of two or more effectors is at least 25 percent greater than the combination's additive

### DETAILED DESCRIPTION OF THE INVENTION

The present Invention relates to a method of treating abnormal cell growth in a subject, comprising administering to said subject having abnormal cell growth: (a) a compound selected from the group consisting of a camptothecin, a camptothecin derivative, an indolopyrrocarbazole derivative, or a pharmaceutically acceptable salt, solvate or prodrug of said compounds; (b) a pyrimidine derivative or a pharmaceutically acceptable salt, solvate or prodrug of said pyrimidine derivative; and (c) an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens and combinations thereof.

Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays α-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with (a) a compound selected from the group consisting of a camptothecin, a camptothecin derivative, an indolopyrrocarbazole derivative, or a pharmaceutically acceptable salt, solvate or prodrug of said compounds; and (b) a pyrimidine derivative or a pharmaceutically acceptable salt, solvate or prodrug of said pyrimidine derivative.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

In one embodiment the Camptothecin is a plant alkaloid obtained from the Chinese tree Camptotheca acuminate.

In one embodiment of the present invention the anti-tumor agent is a kinase inhibitor, pan kinase inhibitor or growth factor inhibitor.

Preferred pan kinase inhibitors include SU-11248, described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase 11) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron,/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following United States patent applications: 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts. GARF inhibitors useful in the practice of the present invention are disclosed in U.S. Pat. No. 5,608,082 which is incorporated in its entirety for all purposed.

Examples of useful COX-II inhibitors include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-11 inhibitors are disclosed in U.S. patent application Ser. Nos. 10/801,446 and 10/801,429, the contents of which are incorporated in their entirety for all purposes.

In one preferred embodiment the anti-tumor agent is celecoxib as disclosed in U.S. Pat. No. 5,466,823, the contents of which are incorporated by reference in its entirety for all purposes. The structure for Celecoxib is shown below:

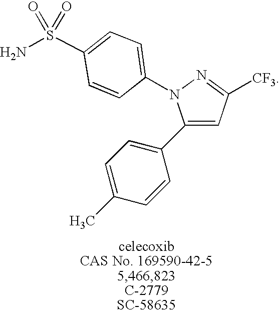

In one preferred embodiment the anti-tumor agent is valecoxib as disclosed in U.S. Pat. No. 5,633,272, the contents of which are incorporated by reference in its entirety for all purposes. The structure for valdecoxib is shown below:

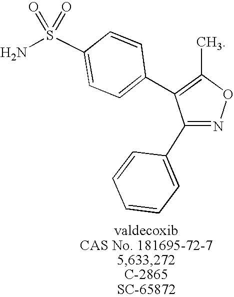

In one preferred embodiment the anti-tumor agent is parecoxib as disclosed in U.S. Pat. No. 5,932,598, the contents of which are incorporated by reference in its entirety for all purposes. The structure for paracoxib is shown below:

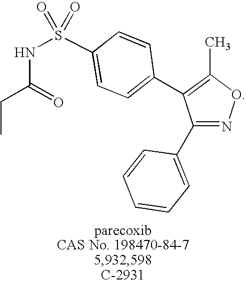

In one preferred embodiment the anti-tumor agent is deracoxib as disclosed in U.S. Pat. No. 5,521,207, the contents of which are incorporated by reference in its entirety for all purposes. The structure for deracoxib is shown below:

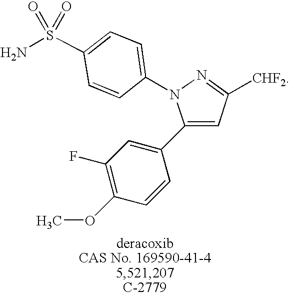

In one preferred embodiment the anti-tumor agent is SD-8381 as disclosed in U.S. Pat. No. 6,034,256, the contents of which are incorporated by reference in its entirety for all purposes. The structure for SD-8381 is shown below:

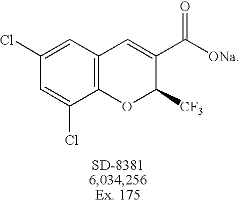

In one preferred embodiment the anti-tumor agent is ABT-963 as disclosed in International Publication Number WO 2002/24719, the contents of which are incorporated by reference in its entirety for all purposes. The structure for ABT-963 is shown below:

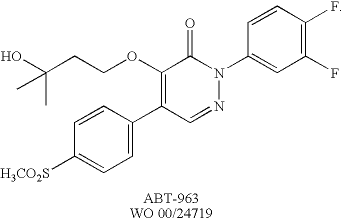

In one preferred embodiment the anti-tumor agent is rofecoxib as shown below:

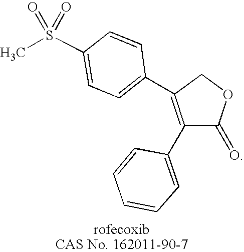

In one preferred embodiment the anti-tumor agent is MK-663 (etoricoxib) as disclosed in International Publication Number WO 1998/03484, the contents of which are incorporated by reference in its entirety for all purposes. The structure for etoricoxib is shown below:

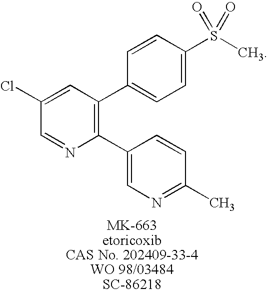

In one preferred embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

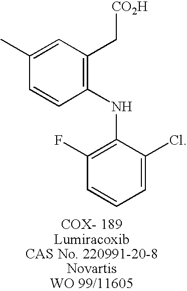

In one preferred embodiment the anti-tumor agent is BMS-347070 as disclosed in U.S. Pat. No. 6,180,651, the contents of which are incorporated by reference in its entirety for all purposes. The structure for BMS-347070 is shown below:

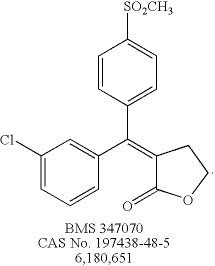

In one preferred embodiment the anti-tumor agent is NS-398 (CAS 123653-11-2). The structure for NS-398 (CAS 123653-11-2) is shown below:

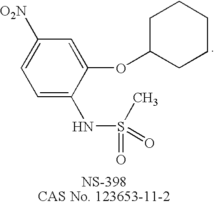

In one preferred embodiment the anti-tumor agent is RS 57067 (CAS 17932-91-3). The structure for RS-57067 (CAS 17932-91-3) is shown below:

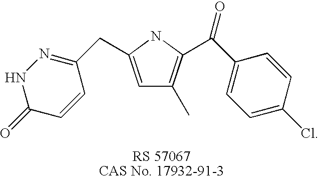

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

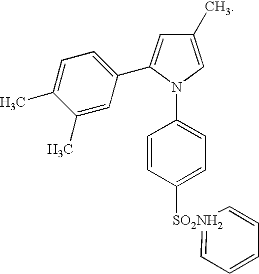

In one preferred embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

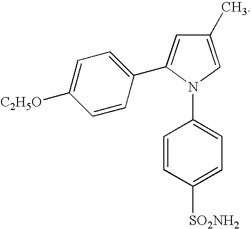

In one preferred embodiment the anti-tumor agent is meloxicam. The structure for meloxicam is shown below:

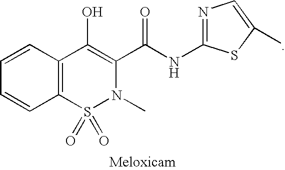

Other useful inhibitors as anti-tumor agents include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof.

Preferred EGFr inhibitors include Iressa, Erbitux, Tarceva and combinations thereof.

The present invention also relates to anti-tumor agents selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aetema), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paditaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (trimetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (131I ch TNT 1/b), NBI-3001 (IL-4) and combinations thereof.

Additional anti-tumor agents may selected from the following agents, Canvaxin, GMK vaccine, Oncophage (HSPPC-96), PEG Interon A, Taxoprexin (DHA/paciltaxel) and combinations thereof.

Other preferred anti-tumor agents include Pfizer's MEK1/2 inhibitor PD325901, Array Biopharm's MEK inhibitor ARRY-142886, Bristol Myers'CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents may be utilized in the present invention, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents camptothecin, irinotecan HCl (Camptosar), edotecarin, orathecin (Supergen), exatecan (Daiichi), BN-80915 (Roche) and combinations thereof.

Particularly preferred toposimerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof.

Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotcs but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9 aminocamptothecin, irinotecan HCl (Camptosar), edotecarin, epirubicin (Ellence), etoposide, SN-38, topotecan, and combinations thereof.

Immunologicals include interferons and numerous other immune-enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, I-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety.

Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety.

Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R, 3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R, 3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;
   and pharmaceutically acceptable salts, solvates and prodrugs of said compounds.

Various other compounds, such as styrene derivatives, have also been shown to possess tyrosine kinase inhibitory properties, and some of tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO0/44728 (published Aug. 3, 2000), EP 1029853A1 (published Aug. 23, 2000), and WO01/98277 (published Dec. 12, 2001) all of which are incorporated herein by reference in their entirety.

The present invention relates in a part to irinotecan [1,4′-Bipiperidine]-1′-carboxylic acid-(4S)-4,11-diethyl-3,4,12,14-tetrahydro-4-hydroxy-3,14-dioxo-1H-pyrano[3′,4′:6;7]-indolizino[1,2-b]quinolin-9-yl ester (CAS RN 97682-44-5) is a camptothecin analog and topoisomerase-1 inhibitor derived from a compound, which occurs naturally in the Chinese tree, Camptotheca acuminata. Irinotecan can be prepared following the procedure disclosed in U.S. Pat. No. 4,604,463, European patent No. 835,257 or S. Sawada et al., Chem. Pharm. Bull. 39, 1446 (1991). Irinotecan hydrochloride trihydrate, clinically investigated as CPT-11, is a commercially available compound (Camptosar®; Pfizer, Inc. and Campto®, Aventis). Irinotecan and other analogs of camptothecin represent a new class of cytotoxic chemotherapeutic agents with a unique mechanism of action. These drugs interact specifically with the enzyme topoisomerase 1, a nuclear enzyme playing a pivotal role in DNA transcription, replication and repair, and are known as topoisomerase I inhibitors.

Irinotecan serves as a water-soluble precursor of the lipophilic metabolite SN-38. SN-38 is formed from irinotecan by carboxylesterase-mediated cleavage of the carbamate bond between camptothecin moiety and the dipiperidino side chain. The primary in vivo site of conversion from the parent drug to SN-38 is thought to be the liver, where the carboxylesterases are abundant. However conversion may also occur in other normal tissues and in tumor sites. The mechanism of action of irinotecan or its metabolite SN-38 is due to double-strand DNA damage produced during DNA synthesis when replication enzymes interact with the ternary complex formed by topoisomerase 1, DNA, and either irinotecan or SN-38, preventing the religation of the strand breaks. Mammalian cells cannot efficiently repair these double-strand breaks. In vitro cytotoxicity assays show that the potency of SN-38 relative to irinotecan varies from 2- to 2000-fold. However, the plasma area under the concentration versus time curve (AUC) values for SN-38 are 2% to 8% of irinotecan and SN-38 is 95% bound to plasma proteins compared with approximately 50% bound to plasma proteins for irinotecan. The precise contribution of SN-38 to the activity of irinotecan is thus unknown although it is believed to be the agent that is primarily responsible for the in vivo activity of irinotecan.

Both irinotecan and SN-38 exist in an active lactone form and an inactive hydroxy acid anion form. A pH-dependent equilibrium exists between the 2 forms, such that an acid pH promotes the formation of lactone, while a more basic pH favors the hydroxy acid anion form.

The IV formulation of irinotecan hydrochloride trihydrate (Camptosar®) is already on the market in many countries for the treatment of subjects with metastatic carcinoma of the colon or rectum whose disease has recurred or progressed following 5-FU-based therapy. In addition, the IV formulation of irinotecan is indicated as a component of first-line therapy in combination with 5-FU and leucovorin for subjects with metastatic carcinoma of the colon or rectum.

Preparation of SSM formulations of oral irinotecan are described in detail in International Patent Application No. WO 2001/30351, published May 3, 2001 and alternative formulations for the oral delivery of irinotecan are described in U.S. Pat. No. 6,569,453. both of which are incorporated by reference in their entirety herein. Oral irinotecan hydrochloride trihydrate is preferably formulated in the form of a semi-solid matrix (SSM) formulation in a capsule containing 5 mg, 20 mg, or 50 mg of active drug substance; inactive ingredients include lecithin and lauryl macrogolglycerides are the preferred form. The 5 mg and 20 mg capsules appear as size 2, self-locking hard gelatin capsules of the Licaps® type, with an opaque white body and cap, containing a yellowish waxy mass. The 50 mg capsules appear as size 0, self-locking hard gelatin capsules of the Licaps® type, with an opaque white cap and body containing a yellowish waxy mass. The capsules were provided in high-density polyethylene (HDPE) bottles with childproof tamper-evident plastic screw caps.

This invention also relates to the use of oral camptothecin.

More specifically, the invention relates to anticancer treatments with associations of camptothecin derivatives include but are not limited to 10-hydroxycamptothecin, 9-aminocamptothecin, 9-nitrocamptothecin, irinotecan, irinotecan salt, SN-38, CPT-11, and topotecan and an indolopyrrocarbazole derivative and a pyrimidine derivative.

Pyrimidine derivatives include but are not limited to uracil, thymine, cytosine, methylcytosine and thiamine containing compounds. Examples of such pyrimidine derivatives are capecitabine, gemcitabine (Gemzar) and multi-targeted antifolate (MTA), also known as pemetrexed.

In one preferred embodiment the indolopyrrocarbazole derivative is administered orally. Indolopyrrocarbazole derivatives are described in the following U.S. Pat. Nos. 5,589,365, 5,437,996, 5,643,760, 5,591,842 and 5,668,271 all of which are incorporated by reference in their entirety.

In one preferred embodiment the indolopyrrocarbazole derivative is edotecarin shown below and pharmaceutically acceptable salts therof:

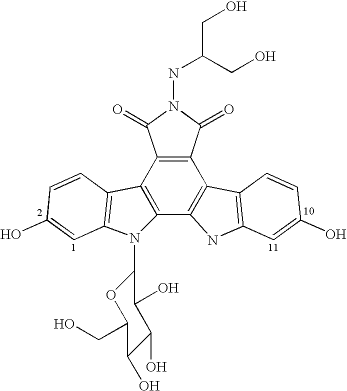

The following U.S. Pat. Nos. 5,804,564 and 5,922,860 described Edotecarin and process for making same, both of the aforementioned patents are incorporated by reference in their entirety.

European patent EP 137,145, incorporated herein, describes camptothecin derivatives of the formula: 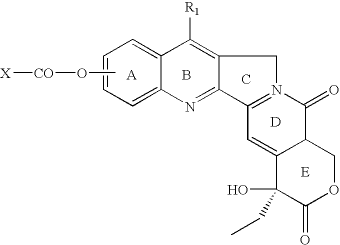
in which, in particular, R₁ is hydrogen, halogen or alkyl, X is a chlorine atom or NR₂, R₃ in which R₂ and R₃, which may be identical or different, may represent a hydrogen atom, an optionally substituted alkyl radical, a carbocycle or a heterocycle which are optionally substituted, or alkyl radicals (optionally substituted) forming, with the nitrogen atom to which they are attached, a heterocycle optionally containing another hetero atom chosen from O, S and/or NR₄, R₄ being a hydrogen atom or an alkyl radical and in which the group X—CO—O— is located in position 9, 10 or 11 on ring A.

These camptothecin derivatives are anticancer agents which inhibit topoisomerase 1, among which irinotecan, in which X—CO—O— is [4-(1-piperidino-1-piperidino]carbonyloxy, is an active principle which is particularly effective in treatment of solid tumors, and in particular, colorectal cancer.

The European patent application EP 74,256 also describes other camptothecin derivatives which are also mentioned as anticancer agents, in particular, derivatives of a structure analogous to the structure given above and in which X—CO—O— is replaced with a radical —X′R′ for which X′ is O or S and R′ is a hydrogen atom or an alkyl or acyl radical.

Other camptothecin derivatives have also been described, for example, in the patents or patent applications EP 56,692, EP 88,642, EP 296,612, EP 321,122, EP 325,247, EP 540,099, EP 737,686, WO 90/03169, WO 96/37496, WO 96/38146, WO 96/38449, WO 97/00876, U.S. Pat. No. 7,104,894, JP 57 116,015, JP 57 116,074, JP 59 005,188, JP 60 019,790, JP 01 249,777, JP 01 246,287 and JP 91 12070 or in Canc. Res., 38 (1997) Abst. 1526 or 95 (San Diego—12-16 April), Canc. Res., 55(3):603-609(1995) or AFMC Int. Med. Chem. Symp. (1997) Abst. PB-55 (Seoul—27 July-1 August).

Camptothecin derivatives are usually administered by injection, more particularly intravenously in the form of a sterile solution or an emulsion. Camptothecin derivatives, however, can also be administered orally, in the form of solid or liquid compositions.

A method that has been used to overcome the poor oral bioavailability of 5-FU involves the administration of a prodrug that has good bioavailability and is ultimately converted to 5-FU. Capecitabine is a fluoropyrimidine antimetabolite considered to act primarily as an inhibitor of thymidylate synthase. Commercially available capecitabine (N⁴-pentyloxycarbonyl-5′-deoxy-5-fluorocytidine, Xeloda®) is supplied as a biconvex, oblong film-coated tablet containing 150 mg or 500 mg of active drug substance; inactive ingredients include lactose, croscarmellose sodium, hydroxypropyl methylcellose, cellulose, magnesium stearate, and water. The 150-mg tablets appear as light peach-colored tablets engraved with XELODA on 1 side and 150 on the other side. The 500-mg tablets appear as peach-colored tablets engraved with XELODA on 1 side and 500 on the other side. The light peach or peach film coating contains hydroxypropyl methycellulose, talc, titanium dioxide, and synthetic yellow and red iron oxides. The capsules will be provided in glass bottles.

Capecitabine is a fluoropyrimidine carbamate with antineoplastic activity. It is an orally administered prodrug of 5′-deoxy-5-fluorouridine (5′-DFUR) which is converted to 5-fluorouracil in the body. Capecitabine has demonstrated activity in colorectal, breast, and head and neck carcinomas, including those resistant to 5-FU.

The chemical name for capecitabine is 5′-deoxy-5-fluoro-N-[(pentyloxy)-carbonyl]-cytidine and it has a molecular weight of 359.35. Capecitabine has the following structural formula:

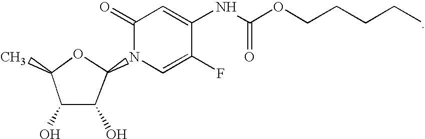

Capecitabine has a unique mechanism of activation that exploits the high concentrations of the enzyme thymidine phosphorylase in tumor tissue compared with healthy tissue, leading to tumor-selective generation of 5-FU. It is readily absorbed from the gastrointestinal tract and is preferentially converted to 5-FU in tumor tissue. After oral administration, capecitabine passes intact from the gastrointestinal tract to the liver, where it is converted by carboxylesterases to 5′-deoxy-5-flourocytidine (5′-DFCR), then by cytidine deaminase in liver and tumor tissue to 5′-deoxy-5-flourouridine (5′-DFUR), and finally by thymidine phosphorylase (dThdPase) in tumor tissue to 5-FU.

Xeloda® (capecitabine, Roche Laboratories, Inc., Nutley, N.J. 07110) is indicated as first-line treatment of patients with metastatic colorectal carcinoma when treatment with fluoropyrimidine therapy alone is preferred. Combination chemotherapy has shown a survival benefit compared to 5-FU/LV. Xeloda® is also approved in combination with docetaxel for the treatment of patients with metastatic breast cancer after failure of prior anthracycline-containing chemotherapy. Xeloda® is also indicated for the treatment of patients with metastatic breast cancer resistant to both paclitaxel and an anthracycline therapy, e.g., patients who have a received cumulative doses of 400 mg/m² of doxorubicin equivalents. Resistance is defined as progressive disease while on treatment, with or without an initial response, or relapse within 6 months of completing treatment with an anthracycline-containing adjuvant regimen.

In one preferred embodiment the treatment cycle is at least 4 weeks, in a more preferred embodiment the treatment cycle is at least 3 weeks.

A new oral formulation of irinotecan in which the drug has been encapsulated as a semi-solid matrix (SSM) has been developed by Pfizer. The SSM capsule is the preferred formulation because of improved safety during manufacturing and handling. The SSM formulation avoids the unintended exposure of the cytotoxic agent to unintended subjects such as family members, pharmacist, and doctors of the patient being treated for cancer.

The bioavailability of the new SSM capsule formulation and the PFC formulation used in prior phase I studies has been compared in dogs. Four dogs were administered 50 mg of each formulation in a crossover design. Blood samples were collected and analyzed for total irinotecan concentrations using HPLC. Mean plasma concentration-time plots following administration of the 2 formulations have been found to be similar. Irinotecan bioavailability was comparable between the new SSM capsule and PFC formulations when studied in dogs.

Both the fluoropyrimidine, 5-fluorouracil (5-FU), and the topoisomerase I inhibitor, CPT-11, are known to be effective antineoplastic agents with wide ranges of tumor activity when administered intravenously (IV). These drugs have become standards of care in the treatment of metastatic colorectal cancer.

The oral administration of cell-cycle-specific agents such as the fluoropyrimidines or CPT-11 is an attractive alternative to IV administration of these types of agents. Oral formulations can achieve protracted drug exposure to actively cycling malignant cells without the need for continuous IV infusion. An oral formulation may offer the advantages of patient convenience and a less expensive means of prolonged drug administration.

In one preferred embodiment of the present invention an oral formulation of camptothecin derivatives, such as irinotecan, permit a convenient method of protracted administration that may be preferred in certain treatment settings in combination with other oral chemotherapeutics. For example, Oral irinotecan and capecitabine combination provide a convenient alternative to IV administration of irinotecan and protracted 5-FU. The present invention relates to the determination of the MTD and DLT of oral irinotecan (semi-solid matrix) formulation given once daily×5 (days 1-5) followed by capecitabine BID daily×9 (days 6-14) q 3 weeks. Additionally, the overall safety profile for the combination has been studied and evidence of antitumor activity for the combination has been found.

Unless otherwise indicated, this disclosure uses definitions provided below.

The term “cancer” includes, but is not limited to, the following cancers: mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

The phrase “pharmaceutically acceptable” refers to substances, which are within the scope of sound medical judgment, suitable for use in contact with the tissues of patients without undue toxicity, irritation, allergic response, and the like, commensurate with a reasonable benefit/risk ratio, and effective for their intended use.

Ligand” is particularly used to describe a small molecule that binds to a receptor. An important class of ligands in the instant invention are those of formula 1 which bind to receptors in the epidermal growth factor family. Ligands can be inhibitors of receptor function and can be antagonists of the action of activators.

Certain abbreviations common in the art are freely used and will be understood in context. Among these are pharmacokinetics (PK), pharmacodynamics (PD), fetal bovine serum (FBS), pennicillin/streptomycin (pen/strep), Roswell Park Memorial Institute (RPMI), per os (PO), once per day (QD), interaperitoneally (IP), subcutaneously (SC), enzyme-linked immunosorbent assay (ELISA), the maximum concentration of an analyte in a PK analysis (Cₘₐₓ), and the average concentration of an analyte in a PK analysis (Cₐᵥₑ).

The term radiation, as used herein, refers to radiation therapy or radiotherapy with ionizing radiation. The radiotherapy may be used locally on a solid tumor, such as brain or breast cancer, or it can also be used to treat cancers of the blood and lymphatic system.

The term “treatment” refers to the act of “treating,” as defined immediately above.

The term “treating” refers to reversing, alleviating, inhibiting the progress of, or preventing a disorder or condition to which such term applies, or to preventing one or more symptoms of such disorder or condition.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

In the present specification “therapeutically effective amount” means, unless otherwise indicated, the amount of drug that is required to be administered to achieve the desired therapeutic effect.

In the present specification the term “sequential” means, unless otherwise specified, characterized by a regular sequence or order.

In the present specification the term “separate” means, unless otherwise specified, to keep apart one from the other.

In the present specification the term “simultaneously” means, unless otherwise specified happening or done at the same time, i.e., the compounds of the present invention are administered at the same time.

In the present specification the term “semi-simultaneously” means, unless otherwise specified means administration of compounds of the present invention at the same time for a period of the treatment regimen, cycle, schedule or course. For example, a non limited example of a semi-simultaneous administration would include the administration of CPT-11 and capecitabine for days 1-5 of a treatment regimen, followed by continued administration of capecitabine for days 6-14, and followed by administration of tarceva for an additional period of days.

In the present specification the term “continuous” means, unless otherwise specified means continuous infusion, by slow release depot, or by injection.

In the present specification the term “regimen” means, unless otherwise specified, refers to: a treatment plan or regimen that specifies the dosage, the schedule, and the duration of treatment (e.g., the specific number of cycles) or for an unspecified number of cycles the duration of the regimen (e.g. until the subject is cured or their disease progresses).

In the present specification the term “cycle” means, unless otherwise specified refers to the period of time (e.g., days) during which a drug is administered to a subject in addition to the drug free days (rest days) until a subsequent treatment cycle is administered to subject. An example of a cycle is as follows: administration of irinotecan once a day on days 1 through 5 followed by administration of capecitabine twice a day on days 6 through 14 followed by drug free days (rest days) 15 through 21. A cycle of treatment with the study drug includes the course of single-agent irinotecan or irinotecan/capecitabine treatment plus the necessary time required for the patient to recover from toxicities, and is expected to be 3 to 5 weeks in duration. Thus, a treatment cycle is defined as the period elapsing from the first day of irinotecan administration for that cycle to Day 22 or Day 29 or Day 36 from the start of the cycle or to the recovery from adverse events sufficient that a new cycle of treatment can be administered, whichever occurs later. If a further cycle of study therapy is initiated even in the absence of these conditions, the prior cycle is considered to be completed.

In the present specification the term “schedule” means, unless otherwise specified refers to the planned sequence, dose and frequency in which chemotherapy drugs are administered each day of each treatment cycle for either a specified number of times (cycles) or until the subject is cured or their disease progresses.

In the present specification the term “course” means, unless otherwise specified refers to the days during each cycle of treatment during which a drug is administered. An example of a course of therapy is the administration of irinotecan on days 1 through 5 (1 course of therapy) followed by the administration on days 6 through 14 of capecitabine (1 course of therapy), followed by drug free days 15 through 21 (1 course).

The terms “cyclooxygenase-2 selective inhibitor” and “COX-2 selective inhibitor” are used interchangeably and refer to a therapeutic compound which selectively inhibits the COX-2 isoform of the enzyme cyclooxygenase. In practice, COX-2 selectivity varies depending on the conditions under which the test is performed and on the inhibitors being tested. However, for the purposes of this patent, COX-2 selectivity can be measured as a ratio of the in vitro IC50 value for inhibition of COX-1, divided by the IC50 value for inhibition of COX-2. A COX-2 selective inhibitor is any inhibitor for which the ratio of COX-1 IC50 to COX-2 IC50 is greater than 1, alternatively greater than 5, in another alternative greater than 10, in yet another alternative greater than 50, and in another alternative greater than 100. In vitro tests useful for determining the COX-1 and COX-2 IC50 values is provided in US. Patent No. 6.034,256, herein incorporated by reference.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The following table 1 provides definitions of abbreviations used in the subject application.

TABLE 1

| Abbreviation | Definition |
|---|---|
| 5′-DFCR | 5′-deoxy-5-flourocytidine |
| 5′-DFUR | 5′-deoxy-5-flourouridine |
| 5-FU | 5-fluorouracil |
| ANC | Absolute neutrophil count |
| Irinotecan | [1,4′-Bipiperidine]-1′-carboxylic acid (4S)-4,11-diethyl- |
| | 3,4,12,14-tetrahydro-4-hydroxy-3,14-dioxo-1H- |
| | pyrano[3′,4′:6,7]indolizino[1,2-b]quinolin-9-yl ester |
| | (CAS RN 97682-44-5) |
| CPT-11 | irinotecan hydrochloride trihydrate, Camptosar ®, |
| | Camptosar ® injection, Campto ® |
| CR | Complete response |
| DLT | Dose-limiting toxicity |
| Dose level | Based on mg/m² of body surface area |
| ECOG | Eastern Cooperative Oncology Group |
| F | absolute bioavailability (fraction absorbed) |
| G-CSF | granulocyte colony-stimulating factor |
| GM-CSF | granulocyte-macrophage colony-stimulating factor |
| HDPE | High-density polyethylene |
| HPLC | High-performance liquid chromatography |
| LV | Leucovorin |
| MTD | maximum tolerated dose |
| NE | not evaluable |
| PD | progressive disease |
| PFC | powder-filled capsule |
| PR | partial response |
| PS | performance status |
| PSA | prostate-specific antigen |
| RECIST | Response Evaluation Criteria in Solid Tumors |
| SD | stable disease |
| SN-38 | active metabolite of CPT-11 |
| SN-38G | SN-38 glucuronide |
| SSM | semi-solid matrix |
| t½ | terminal elimination half-life |
| Tmax | timing of peak plasma concentration |
| ULN | upper limit of normal |
| Vz | volume of distribution |
| WBC | white blood cell count |

The administration of the constituents of the combined preparations of the present invention can be made separately or sequentially in any order. Namely, the present invention intends to embrace administration of camptothecin or camptothecin derivative, such as irinotecan and its pharmaceutically acceptable salts (including CPT-11) and a pyrimidine derivative (e.g., Capecitabine) in a sequential manner in a regimen that will provide beneficial effects of the drug combination, and intends as well to embrace co-administration of these agents within a period of time sufficient to receive a beneficial effect from both of the constituent agents of the combination.

It is therefore another object of the present invention the use of a camptothecin or camptothecin derivative, such as irinotecan and its pharmaceutically acceptable salts (including CPT-11) and a pyrimidine derivative (e.g., Capecitabine) for the preparation of a medicament for sequential use for the treatment of cancer in a patient.

The constituents of the combined preparations according to the invention can be administered to a patient in any acceptable manner that is medically acceptable including orally, parenterally, or with locoregional therapeutic approaches such as, e.g., implants.

Oral administration includes administering the constituents of the combined preparation in a suitable oral form such as, e.g., tablets, capsules, lozenges, suspensions, solutions, emulsions, powders, syrups and the like.

Parenteral administration includes administering the constituents of the combined preparation by subcutaneous, intravenous or intramuscular injections. Implants include intra-arterial implants, for example an intra-hepatic artery implant.

Preferably, camptothecin or camptothecin derivative, such as irinotecan and its pharmaceutically acceptable salts (including CPT-11) may be administered orally in the form of a pharmaceutically acceptable formulation for oral administration, which can provide a means for protracted drug exposure to actively cycling malignant cells with greater convenience and potentially lower costs. In general, the pharmaceutically acceptable formulations for oral administration according to the present invention may comprise a therapeutically effective amount of camptothecin or camptothecin derivative, such as irinotecan and its pharmaceutically acceptable salts (including CPT-11) in combination with a pharmaceutically acceptable carrier or diluent. Examples of oral formulations include solid oral preparations such as, e.g., tablets, capsules, powders and granules, and liquid oral preparations such as e.g., solutions and suspensions, that may be prepared following conventional literature or common techniques well known to those skilled in the art.

Suitable oral dosage forms according to the present invention may be prepared, for example, as described in the Pharmacia & Upjohn S.p.A. International patent application WO 01/10443 filed on Jul. 11, 2000, Teva Pharm. Ind. LTD US patent application No. 20020147208 filed on Dec. 20, 2001 and Pharmacia Italia S.p.A. International patent application WO 01/30351 filed on Oct. 2, 2000.

Preferably, the pyrimidine derivative may be administered orally.

In the method of the subject invention, camptothecin or camptothecin derivative, such as irinotecan and its pharmaceutically acceptable salts (including CPT-11) and a pyrimidine derivative (e.g., Capecitabine) may be administered sequentially, in either order. It will be appreciated that the actual preferred method and order of administration will vary according to, inter alia, the particular formulation of irinotecan being utilized, the particular formulation of revimid being utilized, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. A therapeutically effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped.

In the method according to the present invention, the amount of camptothecin or camptothecin derivative, such as irinotecan and its pharmaceutically acceptable salts (including CPT-11), together with the amount of a pyrimidine derivative (e.g., Capecitabine), constitute an amount therapeutically effective for the treatment of cancer.

A further aspect of the present invention is to provide a method for the treatment of cancer in a patient in need of such a treatment, the method comprising administering to said patient a therapeutically effective amount of camptothecin or camptothecin derivative, such as irinotecan and its pharmaceutically acceptable salts (including CPT-11), together with the amount of a pyrimidine derivative (e.g., Capecitabine).

The dosage regimen should be preferably tailored to the patient's conditions and response and may need to be adjusted in response to changes in conditions.

It has now been found that the sequenced administration of camptothecin derivatives (CPT-11) with pyrimidine derivatives is especially effective in the treatment of solid tumors, such as ovarian, NSCLC and colorectal cancer. Pyrimidine derivatives are that may be used in the present invention include gemcitabine, MTA, and capecitabine. Preferably, the pyrimidine derivative employed is capecitabine.

Gemcitabine (Gemzar) exhibits antitumor activity. The salt of gemcitabine, 2′-deoxy-2′,2′-difluorocytidine monohydrochloride, is provided for clinical use as an intravenous solution for treatment of solid tumors such as non-small cell lung cancer (NSCLC). Gemcitabine exhibits cells phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of gemcitabine is attributed to a combination of two actions of the diphosphate and the triphosphate nucleosides, which leads to inhibition of DNA synthesis. First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is incorporated into DNA. After this addition, there is inhibition of further DNA synthesis.

Gemcitabine has shown promise in combination with CPT-11 as a treatment for pancreatic cancer in Phase II studies.

MTA (multi-targeted antifolate) is an antimetabolite which is a folate antagonist, dihydrofolate reductase inhibitor and thymidylate synthase inhibitor. It is provided for use as an intravenous solution and has been found to inhibit tumor growth in mice. It is currently being tested in humans for treatment of non-small cell lung cancer, mesothelioma, melanoma, bladder cancer, breast cancer, pancreatic cancer, colorectal cancer, and other solid tumors.

For symptoms of diarrhea and/or abdominal cramping that occur at any time during a treatment cycle with single-agent irinotecan or in combination with capecitabine, patients can be treated with Loperamide®. Loperamide should be started at the earliest sign of (1) a poorly formed or loose stool or (2) the occurrence of 1 to 2 more bowel movements than usual in 1 day or (3) a significant increase in stool volume or liquidity. Loperamide should be taken in the following manner: 4 mg at the first onset of diarrhea, then 2 mg every 2 hours around the clock until diarrhea-free for at least 12 hours. Patients may take loperamide 4 mg every 4 hours during the night. Patients should be provided with loperamide, which will be supplied by the sponsor, at the initial treatment visit so that they have sufficient supply on hand in case antidiarrheal support is required. Additional antidiarrheal measures may be used at the discretion of the treating physician. Patients should be instructed to increase fluid intake to help maintain fluid and electrolyte balance during episodes of diarrhea and to record the event in their patient diary.

Prophylactic treatment with antiemetics is not allowed on the first day of treatment in the first course, but can be administered on subsequent treatment days and in subsequent cycles, based on the judgment of the treating physician. The following therapeutic approach is proposed for treatment of nausea and vomiting. At the occurrence of nausea or vomiting of severity grade≧1, it is suggested that the patient receive one of the following agents: (i) thiethylperazine (Torecan®) 10 mg orally 1 to 3 times daily, or (ii) prochlorperazine (Compazine®) 5 or 10 mg orally 3 or 4 times daily, or (ii) metoclopramide (Reglan®) 10-20 mg orally 20 minutes prior to dosing, or (v) chlorpromazine (Thorazine®) 10-25 mg orally every 4 to 6 hours.

If a patient still experiences unacceptable nausea or vomiting with this antiemetic regimen, then the regimen may be changed to include a 5HT3 blocker such as one of the following: (i) ondansetron hydrochloride (Zofran®) 8 mg orally up to 1 hour before irinotecan dosing and up to 2 additional times daily, or (ii) granisetron Hydrochloride (Kytril®) 1 mg orally up to 1 hour before irinotecan dosing, and 1 mg 12 hours later as needed on the day of dosing.

Any other 5HT3-antagonist could be used as preferred by the treating physician instead of those specified above.

In the presence of recurring unacceptable nausea or vomiting additional medications may be employed. Possible agents include: (i) lorazepam (Ativan®) 1 to 2 mg orally every 4 hours, or (ii) dexamethasone (Decadron®) 4 to 8 mg orally twice daily Nausea and vomiting requiring IV antiemetics for prophylaxis should be considered a DLT.

Lacrimation, diaphoresis, flushing, abdominal cramping, diarrhea, or other symptoms of early cholinergic syndrome may occur shortly after taking irinotecan. In past studies, atropine, 0.25-1 mg given intravenously or subcutaneously, has been used as therapy for these symptoms in patients receiving intravenous irinotecan. Bothersome cholinergic symptoms may be treated with oral or sub-lingual hyoscyamine, 0.125-0.25 mg every 4 hours as needed.

Prophylactic administration of G-CSF in a patient who is experiencing recurrent difficulties with neutropenia in subsequent cycles, or therapeutic use in patients with serious neutropenic complications such as tissue infection, sepsis syndrome, fungal infection, etc., may be utilized. Erythropoietin may also be utilized with the present invention.

In the present invention the following definitions in the following table 2 apply to target lesions:

TABLE 2

| RESPONSE | |
|---|---|
| TYPE | |
| FOR TARGET | |
| LESIONS | DEFINITION |
| Complete | Disappearance of all target lesions. |
| response (CR) | |
| Partial | ≧30% decrease in the sum of the longest dimensions of |
| response (PR) | the target lesions taking as a reference the baseline sum |
| | longest dimensions. |
| Progressive | ≧20% increase in the sum of the longest dimensions of |
| disease (PD) | the target lesions taking as a reference the smallest sum |
| | of the longest dimensions recorded since the treatment |
| | started, or the appearance of 1 or more new lesions |
| Stable | Neither sufficient shrinkage to qualify for PR nor |
| Disease (SD) | sufficient increase to qualify for PD taking as a reference |
| | the smallest sum of the longest dimensions since the |
| | treatment started. |

In the present invention the following definitions in the following table 3 apply to non-target lesions:

TABLE 3

| RESPONSE | |
|---|---|
| TYPE FOR | |
| TARGET | |
| LESIONS | DEFINITION |
| Complete | Disappearance of all non-target lesions and |
| response | normalization of tumor marker levels to ≦ULN |
| (CR) | |
| Non- | as a persistence of ≧1 non-target lesions and/or |
| Complete | maintenance of tumor marker levels >ULN |
| Response | |
| (Non- | |
| CR)/Non- | |
| Progressive | |
| disease | |
| (Non-PD | |
| Progressive | unequivocal progression of existing non-target lesions, or |
| disease (PD) | the appearance of ≧1 new lesions |
| Stable | neither sufficient shrinkage to qualify for PR nor sufficient |
| Disease (SD) | increase to qualify for PD taking as a reference the |
| | smallest sum of the longest dimensions since the |
| | treatment started. |

The cytological confirmation of the neoplastic origin of any effusion that appears or worsens during treatment when the measurable tumor has met criteria for response or SD is mandatory to differentiate between response or SD and PD.

To be assigned a status of PR or CR, changes in tumor measurements in patients with responding tumors must be confirmed by repeat studies that should be performed ≧4 weeks after the criteria for response are first met. In the case of SD, follow-up measurements must have met the SD criteria at least once after study entry at a minimum interval of 6 weeks.

When both target and non-target lesions are present, individual assessments will be recorded separately. The overall assessment of response will involve all parameters as depicted in following table 4.

### Response Criteria

TABLE 4

| Target lesions¹ | Non-Target lesions² | New Lesions³ | Overall Response |
|---|---|---|---|
| CR | CR | No | CR |
| CR | Non-CR/Non-PD | No | PR |
| PR | Non-PD | No | PR |
| SD | Non-PD | No | SD |
| PD | Any response | Yes or No | PD |
| Any response | PD | Yes or No | PD |
| Any response | Any response | Yes | PD |

¹Measurable lesions only
²May include measurable lesions not followed as target lesions or non-measurable lesions
³Measurable or non-measurable lesions
Abbreviations:
CR = complete response,
PD = progressive disease,
PR = partial response,
SD = stable disease

The best overall response is the best response recorded from the start of the treatment until disease progression/recurrence (taking as reference for tumor progression the smallest measurements recorded since the treatment started). The patient's best response assignment will depend on the achievement of both measurement and confirmation criteria.

The MTD is the starting dose level at which 0/6 or ⅙ patients experience DLT with the next higher dose having at least ⅔ or 2/6 patients encountering DLT during the first treatment cycle. Effectively the MTD is that dose associated with first-cycle DLT in <33% of patients.

In one aspect of the methods of the invention, the amount of the active agents is at least sufficient to produce therapeutic synergy. In consequence, the combination of the steps of the method of the invention is an improved treatment of a cancer when compared to either alone.

The combination of the invention can be administered orally, buccally, sublingually, vaginally, intraduodenally, parenterally, topically, or rectally. The formulation will preferably be adapted to the particular mode of administration. Antibody combinations of the invention can be administered substantially simultaneously with the other compounds of the combination. The formulations of the individual components of the combination is dependent on the properties of each agent and the desired pharmacological effect desired by the administrator.

The method of the invention is applicable to a human. Non-humans can also be treated. For example, the mammal can be a horse.

The method of the invention is useful for administration to female mammals. The method can also be useful for males. The mammal can be an adult. In another aspect, infants, children, adolescents or the elderly can be treated with the methods of the invention. The methods of the invention are applicable to a wide variety of abnormal cell growth conditions. In one aspect, the methods and kits are advantageously applied to cancers. The cancer can be selected from the group consisting of: mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

Other cancers can also be susceptible to treatment with the methods of the invention. In one aspect, the cancer is selected from the group consisting of ovarian cancer, colon cancer and breast cancer. In another aspect, the cancer is breast cancer or colon cancer. In yet another aspect, the cancer is metastatic breast cancer or colon cancer.

The method of the invention is also applicable to adjuvant therapy, for example, in which the mammal, has received or is receiving a course of chemotherapeutic agents. In such an aspect, the remaining cancer may be a minimal residual disease. In another aspect, the method of the invention can be applied as a prophylactic measure. Thus, for example, the method can be applied to a mammal in cancer remission, in which no measurable disease can be detected.

The invention also comprises a kit comprising: (a) a first agent (camptothecin or camptothecin derivative), as described above, and (b) written instructions packaged with (a), for sequential administration for the treatment of a cancer. Thus, the written instructions can elaborate and qualify the modes of administration.

The invention also comprises a kit comprising: (a) a second agent (pyrimidine derivative), as described above, and (b) written instructions packaged with (a), for sequential administration for the treatment of a cancer. Thus, the written instructions can elaborate and qualify the modes of administration.

The invention also comprises a kit comprising: (a) a first agent (camptothecin or camptothecin derivative) and (b) second agent (pyrimidine derivative), as described above, and (c) written instructions packaged for (a) and (b), for sequential administration for the treatment of a cancer. Thus, the written instructions can elaborate and qualify the modes of administration.

The disclosed compounds may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations. Liquid formulations include suspensions, solutions, syrups and elixirs. Such formulations may be employed as fillers in soft or hard capsules and typically comprise a carrier, for example, water, EtOH, polyethylene glycol, propylene glycol, methylcellulose, or a suitable oil, and one or more emulsifying agents and/or suspending agents. Liquid formulations may also be prepared by the reconstitution of a solid, for example, from a sachet.

The disclosed compounds may also be used in fast-dissolving, fast-disintegrating dosage forms such as those described in Liang and Chen, Expert Opinion in Therapeutic Patents (2001) 11(6):981-986.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methylcellulose, microcrystalline cellulose, lower alkyl-substituted hydroxypropyl cellulose, starch, pregelatinized starch, and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose, and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally include surface-active agents, such as sodium lauryl sulfate and polysorbate 80, and glidants such as silicon dioxide and talc. When present, surface-active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 wt % to 10 wt %, preferably from 0.5 wt % to 3 wt % of the tablet. Other ingredients may include preservatives, anti-oxidants, flavors, and colorants.

Tablet blends may be directly compressed to form tablets. Tablet blends or portions of blends may alternatively be wet-, dry-, or melt-granulated, melt congealed, or extruded before tabletting. The final formulation may comprise one or more layers and may be coated or uncoated. Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant. For additional details concerning the formulation of tablets, see H. Lieberman and L. Lachman, Pharmaceutical Dosage Forms: Tablets, Vol. 1 (1980).

Solid formulations for oral administration may be formulated to be immediate and/or modified release. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14. For a discussion of the use of chewing gum to achieve controlled release, see WO 00/35298.

The disclosed compounds (Formula 1 and salts) may also be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular, and subcutaneous. Suitable devices for parenteral administration include needle (including micro-needle) injectors, needle-free injectors and infusion techniques.

Parenteral formulations are typically aqueous solutions which may contain excipients such as salts, carbohydrates, and buffering agents (preferably to a pH of from 3 to 9), but for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art.

The solubility of the disclosed compounds used in the preparation of parenteral solutions may be increased by the use of appropriate formulation techniques, such as the incorporation of solubility-enhancing agents. Formulations for parenteral administration may be formulated to be immediate and/or modified release as described above. Thus the disclosed compounds may be formulated in a more solid form for administration as an implanted depot providing long-term release of the active compound.

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibers, bandages, and microemulsions. Liposomes may also be used. Typical carriers include alcohol, water, mineral oil, liquid petrolatum, white petrolatum, glycerin, polyethylene glycol and propylene glycol. Topical formulations may also include penetration enhancers. See, for example, Finnin and Morgan, J Pharm Sci (1999) 88(10):955-958.

Other means of topical administration include delivery by iontophoresis, electroporation, phonophoresis, sonophoresis and needle-free (e.g. POWDERJECT) or micro-needle injection. Formulations for topical administration may be formulated to be immediate and/or modified release as described above.

The disclosed compounds can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurized container, pump, spray, atomizer (preferably an atomizer using electrohydrodynamics to produce a fine mist), or nebulizer, with or without the use of a suitable propellant, such as dichlorofluoromethane. The pressurized container, pump, spray, atomizer, or nebulizer contains a solution or suspension, which comprises the active compound, an agent for dispersing, solubilizing, or extending release of the active compound (e.g., EtOH or aqueous EtOH), one or more solvents, which serve as a propellant, and an optional surfactant, such as sorbitan trioleate or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges (made, for example, from gelatin or hydroxypropylmethyl cellulose) for use in an inhaler or insufflator may be formulated to contain a powder mix of the active compound, a suitable powder base such as lactose or starch, and a performance modifier such as L-leucine, mannitol, or magnesium stearate. The lactose may be anhydrous or, preferably, monohydrated. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μl to 100 μl. A typical formulation may comprise a compound of the present invention, propylene glycol, sterile water, EtOH, and NaCl. Alternative solvents, which may be used instead of propylene glycol, include glycerol and polyethylene glycol.

Formulations for inhaled/intranasal administration may be formulated to be immediate and/or modified release using, for example, poly(DL-lactic-coglycolic acid (PGLA). Suitable flavors, such as menthol and levomenthol, or sweeteners, such as saccharin or saccharin sodium, may be added to formulations intended for inhaled/intranasal administration.

In the case of dry powder inhalers and aerosols, the dosage unit is determined by means of a valve that delivers a metered amount. Units in accordance with the invention are typically arranged to administer a metered dose or “puff” containing from 100 to 1000 μg of the active pharmaceutical ingredient. The overall daily dose will typically be in the range 100 μg to 10 mg which may be administered in a single dose or, more usually, as divided doses throughout the day.

The active compounds may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release as described above.

The disclosed compounds may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer (e.g., hydroxypropylmethylcellulose, hydroxyethylcellulose, or methyl cellulose), or a heteropolysaccharide polymer (e.g., gelan gum), may be incorporated together with a preservative, such as benzalkonium chloride. Such formulations may also be delivered by iontophoresis. Formulations for ocular/andial administration may be formulated to be immediate and/or modified release as described above.

The disclosed compounds may be combined with soluble macromolecular entities such as cyclodextrin or polyethylene glycol-containing polymers to improve their solubility, dissolution rate, taste masking, bioavailability and/or stability. Drug-cyclodextrin complexes, for example, are found to be generally useful for most dosage forms and administration routes. Both inclusion and non-inclusion-complexes may be used. As an alternative to direct complexation with the drug, the cyclodextrin may be used as an auxiliary additive, i.e. as a carrier, diluent, or solubilizer. Alpha-, beta- and gamma-cyclodextrins are commonly used for these purposes. See, for example, International Patent Applications WO 91/11172, WO 94/02518, and WO 98/55148.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patents, patent applications, and patent publications, are incorporated herein by reference in their entirety and for all purposes.

### EXAMPLE 1

### Formulation of Oral Irinotecan

The drug product oral irinotecan is supplied in hard gelatin capsules containing 5, 20, or 50 mg as irinotecan hydrochloride trihydrate in a semi-solid matrix.

Composition of the 5, 20, and 50 mg capsules is reported in Table 5.

TABLE 5
Nominal Composition of the Oral Irinotecan Formulation

| | | | | Composition |
|---|---|---|---|---|
| Components | 5 mg | 20 mg | 50 mg | (%) |
| Irinotecan hydrochloride | 5.0 mg | 20.0 mg | 50.0 mg | 7.9 |
| trihydrate (CPT-11) | | | | |
| Lauroyl | 52.4 mg | 209.6 mg | 524.0 mg | 83.2 |
| Macrogolglycerides, | | | | |
| Ph.Eur. (Gelucire) | | | | |
| Lecithin, USP (Epikuron) | 5.6 mg | 22.4 mg | 56.0 mg | 8.9 |
| Total | 63.0 mg | 252.0 mg | 630.0 mg | 100.0 |
| Capsule size | 2 | 2 | 0 | |

NOTE:
It is important to note that the quantitative compositions are exactly proportional, in other words the percent composition is the same for all capsule strengths.

To differentiate the 5, 20, and 50 mg capsules a colored band was applied to the external surface of the capsule shell (ie, the colored band will not be in direct contact with the capsule content), namely:
5 mg dosage, Size 2, self-locking hard gelatin capsules Licaps® type, with an opaque white body and cap.
20 mg dosage, Size 2, self-locking hard gelatin capsules, Licaps® type, with an opaque white body and an opaque white-red printed banded cap.
50 mg dosage, Size 0, self-locking hard gelatin capsules, Licaps® type, with an opaque white body and an opaque white-black printed banded cap.
   The drug product was stored at controlled room temperature in opaque white HDPE bottles, closed with child proof, tamper-evident plastic screw cap.

### EXAMPLE 2

### Method of Administration of Oral Irinotecan and Capecitabine

Irinotecan was administered as a single oral daily dose on days 1-5 of each 3-week cycle of therapy. Irinotecan was administered with water at approximately the same time of each morning and after a fast of 1 hour before and one hour after taking irinotecan. Fasting included abstinence from ingestion of non-investigational prescription or nonprescription medications. Grapefruit juice has been shown to inhibit cytochrome P450 3A4-mediated metabolism of certain drugs in the gut wall [Greenblaft, D, von Moltke, L, Harmatz, J, et al. Time course of recovery of cytochrome P450 3A function after single doses of grapefruit juice. Clinical Pharmacology and Therapeutics 9:74:2 121-129 April, 2003]. Since a component of oral irinotecan metabolism is P450 3A4-mediated, grapefruit juice was not ingested for at least 3 days before or 4 hours after oral irinotecan administration. The appropriate daily dose of irinotecan capsules, based on actual calculated body surface area, was swallowed whole with a glass of tap water (150-200 mL). Day 1 of therapy for each cycle was administered in clinic. Thereafter, patients were given an adequate supply of capsules to take at home for the duration of each single course of treatment.

Capecitabine is commercially available from Roche Labratories, Nutley N.J. 07110 under the brand name Zeloda®. Capecitabine was administered orally as a divided dose, twice daily, on days 6-14 (following administration of oral irinotecan on days 1-5) of each 3-week cycle of therapy. Capecitabine was administered with water (not fruit juices) at approximately the same time each morning and evening, within 30 minutes after a meal, with each dose given approximately 12±2 hours apart. The appropriate dose of capecitabine tablets, based on actual calculated body surface area, was swallowed whole with a glass of tap water (150-200 mL). Patients were given an adequate supply of tablets to take at home for the duration of each single course of treatment.

### EXAMPLE 3

Safety, Pharmacokinetic, and Bioavailability Study of a Semi-Solid Matrix Formulation of Oral Irinotecan and Capecitabine in Patients with Advanced Solid Tumors

Oral irinotecan has the potential to safely and conveniently achieve protracted exposure of cycling tumor cells to SN-38 (irinotecan's active metabolite). The maximum tolerated dose (MTD) of irinotecan SSM was 60 mg/m²/day×5 (Proc ASCO 22:130, 2003 (#521). This study evaluated the maximum tolerated dose (MTD), dose-limiting toxicities (DLT), of oral irinotecan SSM capsules administered on days 1-5 followed by oral capecitabine on days 6-14, followed by a rest period from days 15-21.

Sequential groups of patients received oral irinotecan once daily for 5 consecutive days followed by capecitabine for 9 consecutive days Q3W. MTD was defined as the highest dose level at which less than ⅔ or 2/6 pts experience DLT. 11 additional pts were treated at the MTD. The following table 6 provides a summary of the percentage of grade 3 or 4 adverse events for the study. Significantly no neutropenic fever was reported from the study.

TABLE 6

| Daily Dose | | | | | |
|---|---|---|---|---|---|
| (mg/m²/d) | No of | | | | |
| irinotecan/ | Patients | | | | |
| capecitabine | (N) | Diarrhea | Neutropenia | Nausea | Vomiting |
| 40/1600 | 18 | 1 (5.5%) | 1 (5.5%) | 2 (11%) | 2 (11%) |
| 50/1600 | 3 | 2 (67%) | 0 | 1 (33%) | 0 |

The following study design was employed in the first stage A comprised a dose escalating study to determine the MTD. The second stage of the study Stage B evaluated the feasibility of the study design at MTD for 10 more study subjects. In the study irinotecan was administered orally as a capsule once daily on days 1-5 followed by capecitabine twice daily on days 6-14 for a 3 week study cycle. The capecitabine was administered once in the morning and once in the evening. The following dose levels (mg/m² of body surface area) were employed: (a) 40 mg/m² irinotecan once daily (QD) and 800 mg/m² capecitabine twice daily (BID); and (b) 50 mg/m² irinotecan QD and 800 mg/m² capecitabine BID. The dose escalation was conducted on successive cohorts of 3-6 study subjects. The MTD was defined as highest dose level with less than 2 of 6 dose limiting toxicities (DLTs). The DLT was defined as either of the following adverse events during cycle 1 of the study; (a) hematologic toxicity: grade 4 neutropenia, neutropenic fever, neutropenic infection or grade 4 thrombocytopenia, (b) greater than or equal to grade 3 diarrhea despite maximal loperamide therapy; (c) greater than or equal to grade 2 nausea or vomiting despite maximal antiemetic therapy; (d) greater than or equal to grade 3 non-hematologic toxicities; (e) failure to complete a treatment course; and (f) failure to recover to less than or equal to grade 1 toxicity by day 35.

The following eligibility criteria were use to qualify study subjects (patients) to the study shown in the following table 7. Ineligibility criteria for the study are shown in table 8.

TABLE 7

| ELIGIBILITY CRITERIA | REQUIREMENT |
|---|---|
| Histologically | confirmed solid tumor |
| ECOG PS | 0, 1, or 2 |
| Creatinine | ≦2.0 mg/dl |
| ANC | ≧2,000/μ |
| Platelet | ≧150,000/μ |
| Bilirubin | <ULN |
| AST | ≦3x ULN (≦5x if liver metastases present) |
| Age | ≧18 years |
| Consent | Able to provide informed consent |

TABLE 8

| INELIGIBLE CRITERIA | INELIGIBILITY |
|---|---|
| prior treatment | irinotecan, infusional 5-FU or capecitabine, |
| | mitomycin or nitrosureas |
| Treatment regimens | >2 prior chemo regimens |
| Prior XRT | >25% of bone marrow |
| Prior treatment | Malabsorption, diarrhea, inflammatory bowel |
| related condition | disease, bowel obstruction, total colectomy |
| Tumor involvement | Known CNS tumor involvement |
| Prior treatment | Enzyme inducing anticonvulsants within 2 |
| | week prior to starting therapy |

The following table 9 provides a listing of the characteristics for the patients who participated in this study.

TABLE 9

| STUDY COHORT | | |
|---|---|---|
| TREATMENT | COHORT 1 | COHORT 2 |
| NUMBER OF STUDY | 18 | 3 |
| SUBJECT | | |
| TREATMENT DAYS 1 TO 5 | 40 mg/m² | 50 mg/m² |
| | irinotecan QD | irinotecan QD |
| TREATMENT DAYS 6-14 | 800 mg/m² | 800 mg/m² |
| | capecitabine BID | capecitabine BID |
| TREATMENT DAYS 15-21 | Rest (no drug) | Rest (no drug) |
| AGE | Median 55 | Median 48 |
| | Range 28-78 | Range 32-53 |
| SEX | 8/10 | 2/1 |
| (MALE/FEMALE) | | |
| ECOG PERFORMANCE | 5/11/2 | 1/1/1 |
| STATUS (0/1/2) | | |
| PRIOR CHEMOTHERAPY | Median 1 | Median 2 |
| REGIMENS | Range 0-3 | Range 1-2 |
| TUMOR TYPES | NSCLC (4) | Colorectal |
| *LISTED IN DESCENDING | pancreas (3) | prostate |
| ORDER OF FREQUENCY | kidney (2) | adrenocortical |
| | colorectal (3) | carcinoma |
| | melanoma | |
| | adrenocortical | |
| | cholangiocarcinoma | |
| | fibrosarcoma | |
| | hepatocellular | |
| | Unknown primary | |

The following table 10 shows the dose limiting toxicities observed during the treatment of the patients in cohorts #1 and #2 from table 9.

TABLE 10

| | | | | | Total Cycles |
|---|---|---|---|---|---|
| Dose level | | | | | Adminis- |
| Irinotecan/ | | | Pts | | tered |
| capecitabine | | Evaluable | with | DLT | (median, |
| (mg/m2/day) | Treated | for DLT | DLT | Criteria | range) |
| 40/1600 | 18 | 17 | 2 | Pt 203 | 123 |
| | | | | Gr. 3 Diarrhea | (5, 1-22) |
| | | | | Gr. 3 Vomiting | |
| | | | | Gr. 3 | |
| | | | | Dehydration | |
| | | | | Gr. 3 Nausea | |
| | | | | Pt 219 | |
| | | | | Gr. 3 Vomiting | |
| | | | | Gr. 3 Nausea | |
| 50/1600 | 3 | 3 | 2 | Pt 207 | 5 |
| | | | | Gr. 3 Diarrhea | (2, 1-2) |
| | | | | Gr. 3 | |
| | | | | Dehydration | |
| | | | | Gr. 3 Nausea | |
| | | | | Pt 208 | |
| | | | | Gr. 3 Diarrhea | |

The following table 11 shows the grade 3 and 4 dose limiting toxicities observed during the treatment of the patients in cohorts #1 and #2 from table 9.

TABLE 11

| | | | | | | |
|---|---|---|---|---|---|---|
| Dose level | | | | Vom- | Neutropenic | Neutro- |
| (mg/m²/day) | N | Diarrhea | Nausea | iting | Fever | penia |

Cycle 1

| | | | | | | |
|---|---|---|---|---|---|---|
| 40/1600 | 18 | 1 (6%) | 2 (11%) | 2 (11%) | 0 | 0 |
| 50/1600 | 3 | 2 (67%) | 1 (33%) | 0 | 0 | 0 |

All Cycles

| | | | | | | |
|---|---|---|---|---|---|---|
| 40/1600 | 18 | 1 (6%) | 2 (11%) | 2 (11%) | 0 | 1 (6%) |
| 50/1600 | 3 | 2 (67%) | 1 (33%) | 0 | 0 | 0 |

The following table 12 shows the dose reductions required during the treatment of cohorts #1 and #2 from table 9.

TABLE 12

| DOSE LEVEL | | Number of Cycles | |
|---|---|---|---|
| Irinotecan/capecitabine | | Completed | Cycles with Dose |
| mg/m²/day | N | (range) | Reduced |
| 40/1600 | 18 | 123 | 1 |
| | | (1, 22) | |
| 50/1600 | 3 | 5 | 1 |
| | | (1, 2) | |

The following table 13 shows reason for discontinuation of treatment during the treatment of cohorts #1 and #2 from table 9.

TABLE 13

| | Number of Patients |
|---|---|
| Reason for Discontinuation | (n = 19) |
| Progressive Disease | 15 |
| Adverse Event | 3 |
| Consent Withdrawal | 1 |

The following table 14 shows best response from stage 2 during the treatment of cohorts #1 and #2 from table 9.

TABLE 14

| DOSES | | | | | |
|---|---|---|---|---|---|
| IRINOTECAN/ | | | | | |
| CAPECITABINE | | EVALUABLE | | | |
| (MG/M²/DAY) | N | PATIENTS | PR | SD | PD |
| 40/1600 | 18 | 17 | 2 | 10 | 5 |

The following table 15 shows best tumor response during the treatment of cohorts #1 and #2 from table 9.

TABLE 15

| | Response Type | Number of Evaluable |
|---|---|---|
| Tumor Type | (number of patients) | Patients |
| Colorectal | Partial Response (1) | 4 |
| | Stable Disease (2) | |
| Non-small-cell lung cancer | Partial Response (1) | 4 |
| | Stable Disease (2) | |
| Pancreas | Stable Disease (2) | 3 |
| Melanoma | Stable Disease (1) | 1 |
| Fibrosarcoma | Stable Disease (1) | 1 |
| Hepatocellular | Stable Disease (1) | 1 |
| Adrenocortica | Stable Disease (1) | 1 |

Applicants have discovered that the administration of oral irinotecan as a semi-solid matrix formulation in a capsule in combination with subsequent administration of capecitabine provides for an efficacious method of treating a variety of cancers and provides a convenient alternative to intravenous therapy with irinotecan and 5-FU.

The dosing regimen of oral irinotecan administered once daily×5 q 3 weeks at a dose of 40 mg/m²/day when followed by 9 days of capecitabine given 800 mg/m² BID has been found to be very well tolerated by patients. The favorable safety profile is reflected by a low incidence of adverse events and the limited number of dose reductions (2 out of 128 cycles). Surprisingly, no neutropenic fever was reported during these studies.

It is envisaged that other dose combinations of irinotecan and capecitabine may also be employed such as 50 mg/m²/day irinotecan followed by 800 or 1000 mg/m² BID capecitabine. Additionally, pharmacogenetic evaluation of enrolled patients for UGT1A1 and other genes associated with the metabolism and disposition of irinotecan may be utilized to limit adverse related events.

### EXAMPLE 4

### Oral Treatment Regiment for Cancer Patients

Subjects can be treated by administering dose combinations of irinotecan, capecitabine and anti-tumor agent. Irinotecan is administered once a day at 50 mg/m²/day followed by 800 or 1250 mg/m² BID Capecitabine and an anti-tumor agent, such as, Iressa 250 mg tablet once a day. Alternative, anti-tumor agents may be employed in place of Iressa such as 100 mg or 400 mg Gleevec tablets.

### EXAMPLE 5

### Combination Therapy Treatment Regiment for Cancer Patients

Subjects can be treated by administering dose combinations of irinotecan, capecitabine and anti-tumor agent. Irinotecan is administered once a day at 50 mg/m²/day followed by 800 or 1250 mg/m² BID Capecitabine and an anti-tumor agent, such as, Avastin or Erbitux administered infusionally.

1. A method of treating abnormal cell growth in a subject, comprising administering to said subject having abnormal cell growth: (a) a compound selected from the group consisting of a camptothecin, a camptothecin derivative, an indolopyrrocarbazole derivative, or a pharmaceutically acceptable salt, solvate or prodrug of said compounds; (b) a pyrimidine derivative or a pharmaceutically acceptable salt, solvate or prodrug of said pyrimidine derivative; and (c) an anti-tumor agent selected from the group consisting of antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, anti-androgens and combinations thereof.

2. The method of claim 1, wherein the camptothecin or camptothecin derivative is selected from the group consisting of camptothecin, 10-hydroxycamptothecin, 9-aminocamptothecin, 9-nitrocamptothecin, irinotecan, irinotecan salt, SN-38, CPT-11, topotecan or a pharmaceutically acceptable salt, solvate or prodrug thereof and said indolopyrrocarbazole derivative is edotecarin.

3. The method of claim 2, wherein the camptothecin derivative is selected from the group consisting of irinotecan, SN-38, topotecan or a pharmaceutically acceptable salt, solvate or prodrug thereof.

4. (canceled)

5. (canceled)

6. (canceled)

7. The method of claim 3, wherein the camptothecin derivative is irinotecan hydrochloride trihydrate.

8. (canceled)

9. (canceled)

10. (canceled)

11. (canceled)

12. The method of claim 1, wherein the pyrimidine derivative is selected from the group consisting gemcitabine, multitargeted antifolate (MTA) and capecitabine.

13. (canceled)

14. (canceled)

15. (canceled)

16. (canceled)

17. (canceled)

18. The method of claim 1, wherein the anti-tumor agent is selected from the group consisting of pan kinase inhibitors, growth factor inhibitors, EGF inhibitor, EGFR inhibitors, VEGF inhibitors, VEGFR inhibitors, TIE2 inhibitors, IGF1R inhibitors, erbB2 inhibitors, pan erbB2 inhibitors, CTLA4 monoclonal antibody inhibitors, IGF1R monoclonal antibody inhibitors, CD40 monoclonal antibody inhibitors, MEK inhibitors, pan CDK inhibitors, CDK4 inhibitors, pan AKT inhibitors, TRK inhibitors, anthracycline inhibitors, aromasin inhibitors, topoisomerase I inhibitors, topoisomerase II inhibitors, cox I inhibitors, cox II inhibitors, cytotoxic, and radiation.

19. The method of any of claim 1, wherein the anti-tumor agent is selected from the group consisting of SU-11248, CP-547,632, CP-868,596, CP-724,714, CI-1033, GW-572016, pan erbB2 inhibitor, CTLA4 monoclonal antibody, IGF1R monoclonal antibody, CD40 monoclonal antibody, AG-013736, AG-002037, PD-0332991, PD-0325901, Aromasin® (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Omnitarg, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)₄-methyl-1-(4-sulfamoylphenyl)-1H-pyrrole, T-614, JTE-522, S-2474, SVT-2016, CT-3, SC-58125, Arcoxia (etoricoxib) and radiation.

20. (canceled)

21. (canceled)

22. (canceled)

23. (canceled)

24. (canceled)

25. (canceled)

26. (canceled)

27. (canceled)

28. The method of claim 1, wherein the anti-tumor agent is selected from the group consisting of Tarceva™ (erlotinib HCl) and Avastin™ (bevacizumab).

29. (canceled)

30. The method of claim 1, wherein the anti-tumor agent is SU-11248.

31. (canceled)

32. (canceled)

33. The method of claim 1, wherein said antitumor agent is radiation.

34. (canceled)

35. (canceled)

36. (canceled)

37. (canceled)

38. The method of claim 1, wherein the compounds (a), (b) and (c) are administered simultaneously, semi-simultaneously, separately, or sequentially during a treatment cycle.

39. (canceled)

40. (canceled)

41. The method of claim 1, wherein the abnormal cell growth is cancer is selected from the group consisting of mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

42. (canceled)

43. (canceled)

44. (canceled)

45. (canceled)

46. The method of claim 1, wherein the cancer treatment is administered in the neoadjuvant setting, adjuvant setting, or in the metastatic disease setting.

47. A method of treating cancer in a subject, comprising administering to said subject having cancer oral CPT-11, capecitabine, and an anti-tumor agent selected from the group consisting of SU-11248, CP-547,632, CP-868,596, CP-724,714, CI-1033, GW-572016, pan erbB2 inhibitor, CTLA4 monoclonal antibody, IGF1R monoclonal antibody, CD40 monoclonal antibody, AG-013736, AG-002037, PD-0332991, PD-0325901, Aromasin® (exemstane), Ellence® (epirubicin), Zinecard® (dexrazoxane), Tarceva™ (erlotinib HCl), Iressa™ (genfitinib), Avastin™ (bevacizumab), Erbitux™ (Cetuximab or C225), Herceptin®, Omnitarg, Bexxar, Zevalin, Rituxan, Panitumumab, Taxol® (paclitaxel), Adriamycin® (doxorubicin), CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125, Arcoxia (etoricoxib) and radiation.

48. The method of claim 47, wherein the anti-tumor agent is selected from the group consisting of SU-11248, CP-547,632, CP-868,596, GW572016, Tarceva™ (erlotinib HCl), Avastin™ (bevacizumab), Erbitux™ (Cetuximab or C225), Celebrex® (celecoxib), paracoxib, Herceptin®, Omnitarg, Vioxx®, (rofecoxib), Bextra® (valdecoxib), Arcoxia™ (etoricoxib) and radiation.

49. The method of claim 47, wherein the anti-tumor agent is selected from the group consisting of SU-11248, GW572016, Tarceva™ (erlotinib HCl), Avastin™ (bevacizumab), Erbitux™ (Cetuximab or C225), Herceptin®, and radiation.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. The method of claim 1, wherein 40 to 50 mg/m² of the oral CPT-11 is administered on days 1 to 5 of a three week cycle and 800 to 1250 mg/m² of the capecitabine is administered on days 6 to 14 of the three week cycle.

93. The method of claim 92, wherein the third week of the cycle is drug free.

94. The method of claim 92, wherein the oral CPT-11 is administered once a day.

95. The method of claim 92, wherein the capecitabine is administered twice a day.
